(19) United States
(12) Patent Application Publication
(10) Pub. No.: US 2025/0387050 A1
(43) Pub. Date: Dec. 25, 2025

(54) SYSTEM FOR NON-INVASIVE OPTICAL MEASUREMENT OF BIO-SUBSTANCE IN BIOLOGICAL TISSUE

(71) Applicant: NATIONAL CHENG KUNG UNIVERSITY, Tainan (TW)

(72) Inventors: YU-LUNG LO, Tainan (TW); GUAN-TING WEI, Tainan (TW); YI-SHENG CHEN, Tainan (TW); PO-YU WANG, Tainan (TW)

(21) Appl. No.: 19/244,324

(22) Filed: Jun. 20, 2025

Related U.S. Application Data
(60) Provisional application No. 63/662,400, filed on Jun. 20, 2024.

(51) Int. Cl. A61B 5/1455; A61B 5/00; A61B 5/145; G16H 40/60

(52) U.S. Cl. CPC A61B 5/1455; A61B 5/14532; A61B 5/6843; A61B 5/7246; A61B 5/7257; A61B 5/7267; G16H 40/60; A61B 5/14546; A61B 2562/0238

(57) ABSTRACT

The present invention discloses a system for non-invasive optical measurement of bio-substance in a biological tissue, comprising: a light source generator to generate an incident light to irradiate at a target area on an individual; an optical reader to receive a reflect light returning from the target area; and a processor to run an optical feature analysis model to extract an optical feature information from the reflect light and determine a concentration value of a bio-substance in the target area according to the optical feature information. With analysis based on optical rotation angles and light intensity signals, in combination of pressure effects and time delay compensation, effective features can be selected as input to the optical feature analysis model to improve the accuracy bio-substance level detection.

### CROSS-REFERENCE TO RELATED APPLICATION

This application claims the benefit of U.S. Patent application Ser. No. 63/662,400, filed Jun. 20, 2024. The disclosure of the above application is incorporated herein in its entirety by reference.

### FIELD OF THE INVENTION

The present invention is related to a bio-substance measurement system, and particularly a system using polarization rotator to extract optical features of bio-substance. The present invention also involves a combined use of machine learning and classification model to enhance prediction accuracy of the bio-substance level.

### BACKGROUND OF THE INVENTION

To prevent the progression of diabetes, continuous monitoring of blood glucose levels is required. Conventional invasive methods require puncturing the patient's skin to collect blood samples, causing painful and discomfortable experience to the patients. To date, various non-invasive blood glucose measurement techniques have been developed. Spectroscopic techniques including specular reflection (SR) and diffuse reflection (DR) attenuated total reflection spectroscopy (ATR), photoacoustic (PA), photothermal (PT), and polarimetric methods have been intensively investigated.

Among polarimetric systems, a small, noninvasive blood glucose monitor that utilizes polarized light is developed. A mathematical model based on the Mueller matrix theory is utilized to establish the relationship between these reflected signals and blood glucose levels. Users can measure glucose levels by placing their palms on the device. Techniques to reduce skin light scattering and extract precise glucose estimates from subtle polarization changes have also drawn attentions.

A polarimetric glucose sensor is proposed to utilize a liquid-crystal polarization modulator (LCPM) driven by a sinusoidal signal. This design enhances accuracy and sensitivity in glucose measurement by modulating light polarization according to glucose concentration changes. The sinusoidal signal improves stability and precision, making the sensor effective for real-time, non-invasive glucose monitoring. A Stokes-Mueller matrix polarimetry system is also developed for glucose sensing, which significantly enhances measurement accuracy and sensitivity. This method allows for precise detection of glucose concentration variations by quantifying the alterations in the polarization state of light. Experimental results validate the system's effectiveness in reliable, non-invasive glucose monitoring.

Another noninvasive blood glucose measurement method using polarimetric techniques and partial least squares regression (PLSR) is also developed by leveraging the principles of polarimetry to analyze the rotation of light through biological tissues. Changes in polarimetric data are correlated with glucose concentrations in blood. The integration of PLSR allows the development of predictive models that enhance the accuracy of glucose measurements based on polarimetric data. In addition, advanced 3D laser Müller matrix polarimetry methods have been established by employing phase scanning to reconstruct the optical anisotropy parameters of myocardial histopathology tissue samples. These methods effectively eliminate the influence of multiply scattered depolarizing backgrounds within the tissue volume, demonstrating efficiency and sensitivity of non-invasive glucose sensing within the tissue volume.

### SUMMARY OF THE INVENTION

The present invention discloses a system to extract bio-substance level in a biological tissue based on uniformly processed optical feature data harvested via a single polarization rotator system. The optical feature data are fed to a classification model for regression training so as to address the issue of data imbalance caused by significant fluctuations in bio-substance levels among diabetic patients.

In one aspect, the present invention provides a system for non-invasive optical measurement of bio-substance in a biological tissue, comprising: a light source generator, configured to generate an incident light to irradiate at a target area on an individual; an incident polarizer set, positioned on an incident light optical path of the incident light, and being configured of a 0-degree polarizer and a rotating polarizer in a sequential manner, wherein: the rotating polarizer spins about the incident light optical path at an angular velocity; or the rotating polarizer spins about an axis at another angular velocity, wherein the axis forms an acute angle with the incident light optical path; an optical reader, configured to receive a reflect light returning from the target area; a reflect polarizer set, positioned on a reflect light optical path of the reflect light, and being configured of a 90-degree polarizer; and a processor, signally connected to the light source generator and the optical reader, and being configured to run an optical feature analysis model to extract an optical feature information from the reflect light and to determine a concentration value of a bio-substance in the target area according to the optical feature information.

In another aspect, the present invention provides a system for non-invasive optical measurement of bio-substance in a biological tissue, comprising: a light source generator, configured to generate an incident light to irradiate at a target area on an individual; an incident polarizer set, positioned on an incident light optical path of the incident light, and being configured of a 0-degree polarizer and a rotating polarizer in a sequential manner, wherein: the rotating polarizer spins about the incident light optical path at an angular velocity; or the rotating polarizer spins about an axis at another angular velocity, wherein the axis forms an acute angle with the incident light optical path; an optical reader, configured to receive a reflect light returning from the target area; a reflect polarizer set, positioned on a reflect light optical path of the reflect light, and being configured of a 90-degree polarizer; and a processor, signally connected to the light source generator and the optical reader, wherein: in a training phase, the processor is configured to run a pre-optical feature analysis model to retrieve a plurality of optical feature information and a plurality of reference concentration information, and train the pre-optical feature analysis model into an optical feature analysis model based on the plurality of the optical feature information and the plurality of the reference concentration information.

### BRIEF DESCRIPTION OF THE DRAWINGS

FIGS. 1A to 1C are schematic diagrams of the system in the first aspect of the present invention;

FIGS. 2A to 2C are flowcharts to illustrate working mechanism of the optical feature analysis model;

FIG. 3 is a schematic diagram of the system in the second aspect of the present invention;

FIGS. 4A to 4C are flowcharts to illustrate pipeline of the pre-optical feature analysis model in training phase;

FIG. 4D is a flowchart to illustrate pipeline of the optical feature analysis model in training phase;

FIGS. 5A to 5B show Random Forest training and predict results in transmission mode simulation;

FIG. 6 is a heatmap to show feature correlation in a phantom experiment using transmission mode;

FIGS. 7A to 7B show Random Forest training and predict results in a phantom experiment using transmission mode;

FIG. 8 shows consecutive measurements of reflect light intensity with 300-gram weight applied on finger for 2 minutes;

FIG. 9 shows consecutive measurements of reflect light intensity with 400-gram weight applied on finger for 2 minutes;

FIG. 10 shows the stability signals obtained from two consecutive measurements on the human body within the same period;

FIG. 11 is a heatmap to show feature correlation in a human experiment using transmission mode;

FIGS. 12A to 12B show Random Forest training and predict results in a human experiment without compensating time delay;

FIGS. 13A to 13B show Random Forest training and predict results in a human experiment with compensating time delay;

FIGS. 14A to 14B show Random Forest training and predict results with skewed distribution of imbalanced data from diabetic participants;

FIGS. 15A to 15B show Random Forest training and predict results after the imbalanced data were weight-adjusted;

FIGS. 16A to 16B represent Clarke Error Grid analysis of data from healthy individuals;

FIGS. 17A to 17B represent Clarke Error Grid analysis of data from diabetic patients;

FIGS. 18 to 19 show correlation coefficients of the Fourier Expansion coefficients and glucose concentration in reflection mode simulation;

FIG. 20 shows Random Forest training and predict results in reflection mode simulation;

FIG. 21 shows Random Forest training and predict results in a phantom experiment using reflection mode;

FIGS. 22A to 22B shows the prediction results with or without using the Stepwise Regression method;

FIGS. 23A and 23B represent Clarke Error Grid analysis of data from healthy participants.

FIGS. 24A and 24B represent Clarke Error Grid analysis of data from diabetic participant.

### DETAILED DESCRIPTION OF THE INVENTION

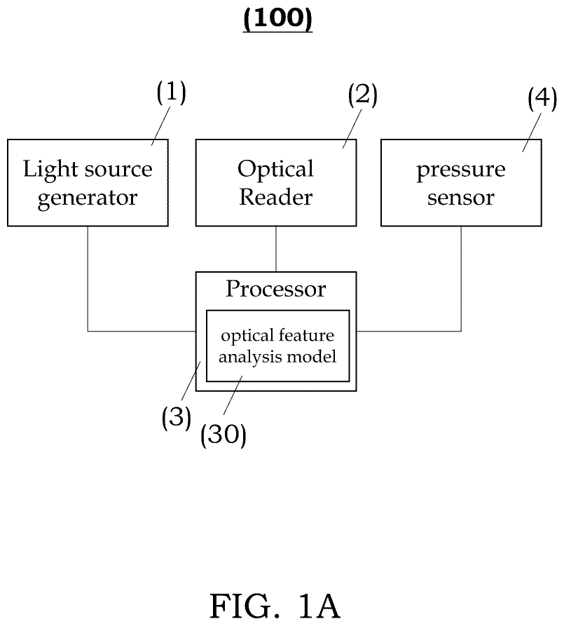

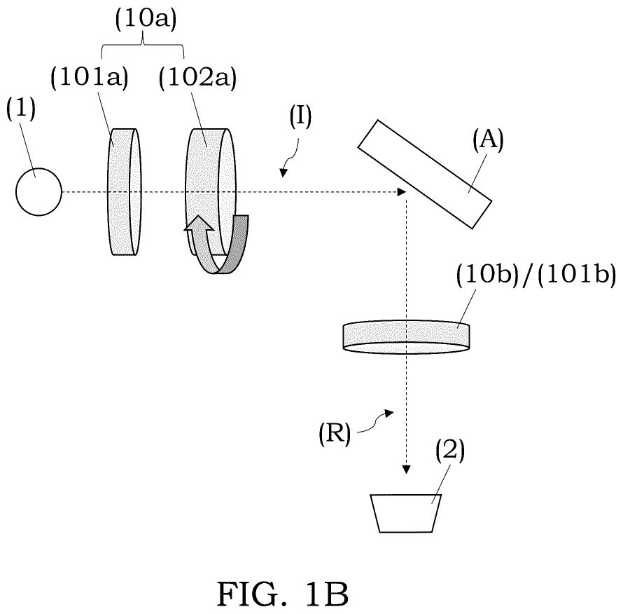

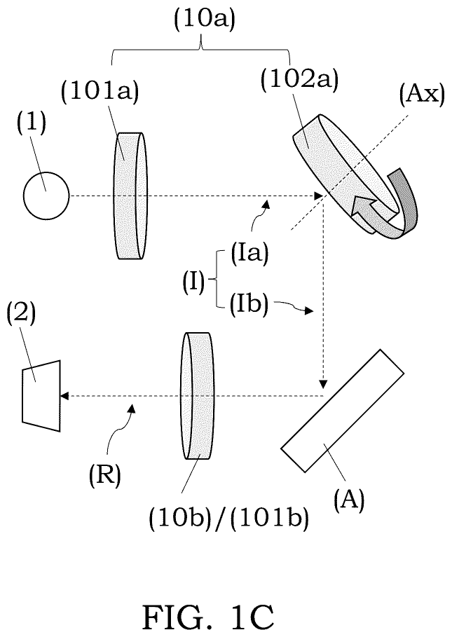

Please refer to FIG. 1A, provided in the first aspect of the present invention is a system (100) for non-invasive optical measurement of bio-substance in a biological tissue, comprising a light source generator (1), an optical reader (2) and a processor (3), wherein the processor (3) is configured of an optical feature analysis model (30) for processing optical feature information to determine bio-substance concentration in a target area of an individual.

The light source generator (1) is configured to generate an incident light to irradiate at a target area on an individual. The light source generator (1) may use a LED light source, a fluorescent light source or a laser light source. As for LED light source or laser light source, the working spectrum spans from Near-Infrared (NIR), Mid-Infrared (MIR) or visible light. In preferred embodiments, the light source generator (1) uses a laser light source, and the incident light is generated of wavelength from 620 to 750 nm, and 660 nm red laser beam is more preferable.

The target area (A) may be defined as a portion on a skin surface of an individual where a blood vessel lies thereunder, or a biological tissue model. The skin surface may be on an abdomen, a forearm, an upper arm, a thigh, a calf, a palm, a fingertip, or foot toe of the individual, but not limited to this. The individual may be a human or other mammals such as mouse, pig, dog, sheep, cow or cat. In preferred embodiment, the individual is a human, and particularly with a human diabetic patient.

The bio-substance comprises blood sugar, cholesterol, lipid, protein uric acid, or any other type of bio-substance circulating in the blood vessel. Conceivably, the light source generator (1) may switch a different type of light source depending on the specific species of the bio-substance. For example, the bio-substance is blood sugar, considering light absorbance, a laser of 660 nm may be chosen.

The optical reader (2) is configured to receive a reflect light returning from the target area (A).

FIGS. 1B to 1C illustrates the system (100) configured of an incident polarizer set (10a) and a reflect polarizer set (10b), respectively. Generally, the incident polarizer set (10a) is positioned on an incident light optical path (I) of the incident light. The incident polarizer set (10a) includes a 0-degree polarizer (101a) and a rotating polarizer (102a) arranged in a sequential manner along the incident light optical path (I). The reflect polarizer set (10b) is positioned on a reflect light optical path (R) of the reflect light. The reflect polarizer set (10b) includes a 90-degree polarizer (101b).

In some embodiments, the system (100) is configured in a transmission mode as shown in FIG. 1B. In a direction from the light source generator (1) towards the target area (A), the 0-degree polarizer (101a) and the rotating polarizer (102a) are arranged sequentially along the incident light optical path (I). Specifically, the incident light travels from the light source generator (1), passing through the 0-degree polarizer (101a) and the rotating polarizer (102a) sequentially, thereby reaching the target area (A) so that another light beam travels from the target area (A) as the reflect light. The reflect light then passes through the 90-degree polarizer and reaches the optical reader (2). In transmission mode, the rotating polarizer (102a) spins about the incident light optical path (I) at an angular velocity.

In some other embodiments, the system (100) is configured in a reflection mode as shown in FIG. 1C. As the rotating polarizer (102a) spins about an axis (Ax) at another angular velocity, wherein the axis (Ax) forms an acute angle with the incident light optical path (I), the incident light is polarized and further redirected to the target area (A). in preferred embodiments, the acute angle is 30 to 60 degrees, and preferably 40 to 50 degrees, exemplarily, the acute angle may be 40, 41, 42, 43, 44, 45, 46, 47, 48, 49 or 50 degrees.

Considering to redirect the incident light to the target area (A), the rotating polarizer further comprises a reflect coating on one surface thereof. To be clear, in reflection mode, incident light may be considered to be a combination of a first incident light and a second incident light. The first incident light hits the rotating polarizer, and reaches the reflect coating, thereby reflects from the reflect coating to become, being polarized, the second incident light.

In this case, one can imagine that the incident light optical path (I) may be a combination of a first optical path (Ia) and a second optical path (Ib), wherein the first optical path (Ia) connects the light source generator (1) and the rotating polarizer (102a) and along which the first incident light travels; the second optical path (Ib) connects the rotating polarizer (102a) and the target area (A) and along which the second incident light travels. The second incident light reaches the target area (A) and turns to be the reflect light travelling away from the target area (A). The reflect light is then polarized by the 90-degree polarizer (101b) before reaching the optical reader (2).

The processor (3) is signally connected to the light source generator (1) and the optical reader (2), and configured to run the optical feature analysis model (30). In various embodiments, the processor (3) is configured to receive an acoustic signal, an electrical signal or an optical signal from the optical reader (2), depending on the signal transformation mechanism in the optical reader (2). For example, the acoustic signal may be generated when the optical reader (2) read the reflect light and triggers on a built-in oscillator, wherein the acoustic signal includes audio sound signal or ultrasonic sound signal; the electrical signal may be a digital signal or an analog signal, and the electrical signal may be generated by an optoelectrical component or a photo transistor; the optical signal may be transmitted from the optical reader (2) to the processor (3) via an optical fiber.

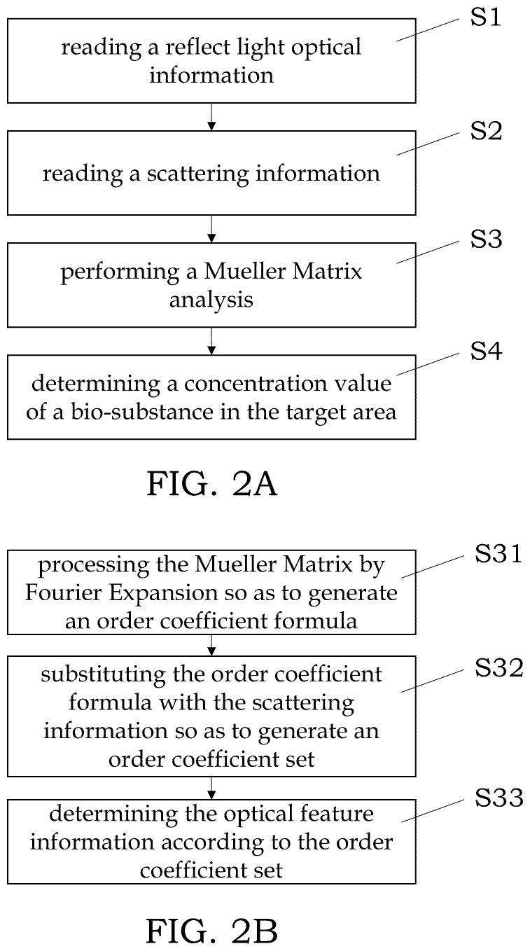

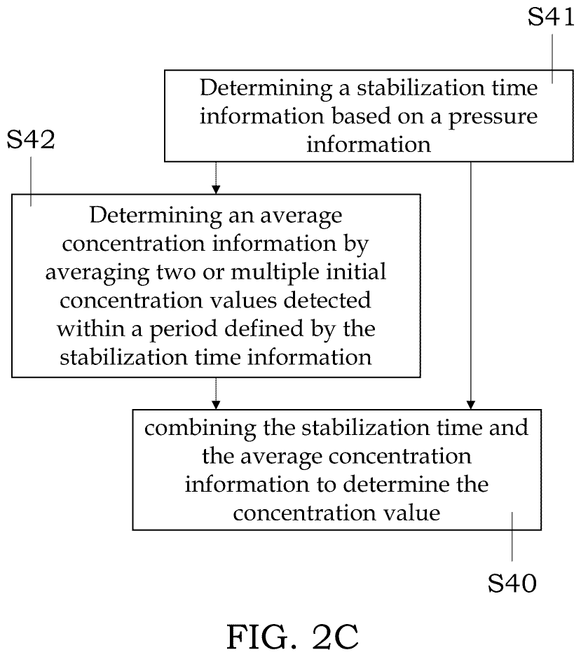

FIGS. 2A to 2C illustrate the pipeline of the optical feature analysis model (30) to determine the bio-substance concentration based on the optical feature information.

As shown in FIG. 2A, the optical feature analysis model (30) executes the following steps:
Step S1: reading a reflect light optical information corresponding to the reflect light, wherein the reflect light optical information comprising a reflect light intensity and a reflect light polarization information;
Step S2: reading a scattering information corresponding to the target area (A), wherein the scattering information comprises one or multiple depolarization factors;
Step S3: performing a Mueller Matrix analysis based on the reflect light intensity and reflect light polarization information so as to obtain an optical feature information, wherein the optical feature information comprises a reflect light polarization feature and target area scattering feature; and
Step S4: determining a concentration value of a bio-substance in the target area (A) according to the optical feature information.

The Mueller Matrix analysis is performed based on an incident light information corresponding to the incident light, wherein the incident light information comprises a Stokes vector information, an incident light polarization information and a rotation information.

As shown in FIG. 2B, the optical feature analysis model (30) is configured to further execute Mueller Matrix analysis following steps of:
Step S31: processing the Mueller Matrix by Fourier Expansion so as to generate an order coefficient formula (Eq. 3) corresponding to the reflect light intensity; and
Step S32: substituting the order coefficient formula with the scattering information so as to generate an order coefficient set; and
Step S32: determining the optical feature information according to the order coefficient set.

Hereinafter, the Mueller Matrix analysis is illustrated in details based on the system (100) in the transmission mode and the reflection mode, respectively.

### Transmission Mode

The Mueller Matrix analysis may be expressed as a Stokes vector-Mueller matrix equation which is formulated as equation (1):

$${S}_{out} = {M}_{polarizer , 90 °} \cdot {M}_{sample ( γ )} \cdot {M}_{scatter} \cdot {M}_{sample ( γ )} \cdot {M}_{rotator} \cdot {M}_{polarizer , 0} \cdot {S}_{in} \qquad ( 1 )$$

Where each of the parameters is defined as the followings:
Sᵢₙ represents the Stokes vector information of incident light, and the Stokes vector of laser rays are exemplified;
M_{polarizer, 0°} represents the incident light polarization information, and exemplified by a Mueller matrix of the 0-degree polarizer (101a);
Mᵣₒₜₐₜₒᵣ represents the rotation information, and exemplified by a Mueller matrix of the rotating polarizer (102a) at a rotating frequency @;
M_{sample(γ)} represents a sample optical rotation angle information, and exemplified by an optical rotation Mueller matrix generated in reference with the concentration change of the bio-substance at the target area (A);
M_{scatter} represents a Mueller matrix of the scattering information, and exemplified by simulated scattering from the target area (A), including the one or multiple depolarization factors;
M_{polarizer, 90°} represents the reflect light polarization information, and exemplified by a Mueller matrix of the 90-degree polarizer.
Sₒᵤₜ represents the reflect light intensity, namely, the final output ray.

In view of the above, Equation (2) may be rewritten to incorporate all these Mueller matrices and vectors, and reformulated as equation (2):

$$[ \begin{matrix}{S}_{out 0} \\ {S}_{out 1} \\ {S}_{out 2} \\ {S}_{out 3}\end{matrix} ] = \frac{1}{2} [ \begin{matrix}- 1 & - 1 & 0 & 0 \\ - 1 & 1 & 0 & 0 \\ 0 & 0 & 0 & 0 \\ 0 & 0 & 0 & 0\end{matrix} ] \cdot [ \begin{matrix}1 & 0 & 0 & 0 \\ 0 & cos ( 2 γ ) & sin ( 2 γ ) & 0 \\ 0 & - sin ( 2 γ ) & cos ( 2 γ ) & 0 \\ 0 & 0 & 0 & 1\end{matrix} ] \cdot [ \begin{matrix}1 & 0 & 0 & 0 \\ 0 & {D}_{1} & 0 & 0 \\ 0 & 0 & {D}_{2} & 0 \\ 0 & 0 & 0 & {D}_{3}\end{matrix} ] \cdot [ \begin{matrix}1 & 0 & 0 & 0 \\ 0 & cos ( 2 γ ) & sin ( 2 γ ) & 0 \\ 0 & - sin ( 2 γ ) & cos ( 2 γ ) & 0 \\ 0 & 0 & 0 & 1\end{matrix} ] \cdot \frac{1}{2} [ \begin{matrix}1 & cos ( 2 ω t ) & sin ( 2 ω t ) & 0 \\ cos ( 2 ω t ) & {cos}^{2} ( 2 ω t ) & sin ( 2 ω t ) \cdot cos ( 2 ω t ) & 0 \\ sin ( 2 ω t ) & cos ( 2 ω t ) sin ( 2 ω t ) & {sin}^{2} ( 2 ω t ) & 0 \\ 0 & 0 & 0 & 0\end{matrix} ] \cdot \frac{1}{2} [ \begin{matrix}1 & 1 & 0 & 0 \\ 1 & 1 & 0 & 0 \\ 0 & 0 & 0 & 0 \\ 0 & 0 & 0 & 0\end{matrix} ] \cdot [ \begin{matrix}{S}_{in 0} \\ {S}_{in 1} \\ {S}_{in 2} \\ {S}_{in 3}\end{matrix} ] \qquad ( 2 )$$

Where each of the parameters is defined as the followings:
ω represents the angular velocity of the rotating polarizer (102a);
γ represents the optical rotation angle, and γ variates with types of the bio-substance or concentration levels of the bio-substance;
D₁, D₂, D₃ in the M_{scatter} represent the one or multiple depolarization factors, and all of one or multiple depolarization factors are positive values.

In this case, the first element of the Stokes vector is the reflect light intensity, namely the Sₒᵤₜ, and mainly the total intensity I extracted by the optical reader (2). Therefore, equation (1) may be further extended and equation (2) is rewritten into a Fourier expansion form, and Sₒᵤₜ₀ can be expressed as equation (3):

$$I = {S}_{out 0} = {a}_{0} + {\sum }_{n = 1}^{∞} ( {a}_{n} cos ( 2 n ω t ) + {b}_{n} sin ( 2 n ω t ) ) = - \frac{( {D}_{1} - {D}_{2} ) - 4}{16} - \frac{( {D}_{1} - {D}_{2} ) cos ( 4 γ )}{16} + \frac{\{ 2 - {D}_{1} + {D}_{2} - [ ( {D}_{1} + {D}_{2} ) cos ( 4 γ ) ] \} cos ( 2 ω t )}{8} - \frac{( {D}_{1} + {D}_{2} ) sin ( 4 γ ) sin ( 2 ω t )}{8} - \frac{\{ {D}_{1} - {D}_{2} + [ ( {D}_{1} + {D}_{2} ) cos ( 4 γ ) ] \} cos ( 4 ω t )}{16} - \frac{( {D}_{1} + {D}_{2} ) sin ( 4 γ ) sin ( 4 ω t )}{16} \qquad ( 3 )$$

Based on the equation (3), as a Fourier expansion equation, the Fourier expansion coefficients can be determined. In equation (3), each value of D represents each of the one or multiple depolarization factors D₁, D₂, D₃, and each one depolarization factor is a constant value as the angle of the incident light hitting the target area (A) is fixed.

Particularly, the depolarization factor may be a 40-degree depolarization factor or a 45-degree depolarization factor. In preferred embodiments, the Mueller matrix of a 40-degree depolarization factor, as illustrated in equation (4), is introduced. The depolarization factors from equation (4) are substituted into equation (3), and the order coefficient set are obtained as shown in Table 1. The reflect light polarization feature and the target area scattering feature can be both described in mathematic form.

$${M}_{scatter} = [ \begin{matrix}1 & 0 & 0 & 0 \\ 0 & 0.8 & 0 & 0 \\ 0 & 0 & 0.4 & 0 \\ 0 & 0 & 0 & 0.475\end{matrix} ] \qquad ( 4 )$$

TABLE 1

| reflect light | target area |
|---|---|
| polarization feature | scattering feature |
| ${a}_{0} = \frac{9}{40} - \frac{3 cos 4 γ )}{40}$ | ${b}_{1} = - \frac{3 sin ( 4 γ )}{20}$ |
| ${a}_{1} = \frac{4 - cos ( 4 γ )}{20}$ | ${b}_{2} = - \frac{3 sin ( 4 γ )}{40}$ |
| ${a}_{2} = - \frac{1 + 3 cos ( 4 γ )}{40}$ | |

### Reflection Mode

The Mueller Matrix analysis may be expressed as a Stokes vector-Mueller matrix equation which is formulated as equations (5.1) and (5.2):

$${S}_{out} = {M}_{polarizer , 90 °} \cdot {M}_{sample ( γ )} \cdot {M}_{scatter} \cdot {M}_{sample ( γ )} \cdot {M}_{Al rotator} \cdot {M}_{polarizer , 0 °} \cdot {S}_{in} \qquad ( 5.1 )$$

$${M}_{Al rotator} = {M}_{polarizer , 90 °} \cdot {M}_{rotator} \cdot {M}_{mirror} \cdot {M}_{polarizer , 0 °} \cdot {M}_{rotator} \qquad ( 5.2 )$$

Where each of the parameters is defined as the followings:
Sᵢₙ, M_{sample(γ)}, M_{scatter}, M_{polarizer, 90°}, Mᵣₒₜₐₜₒᵣ and Sₒᵤₜ have been previously defined in section “Transmission Mode”, while M_{Al rotator} is the Mueller matrix of the reflect coating and exemplified by aluminum coating, and Mₘᵢᵣᵣₒᵣ is the Mueller matrix of the mirror.

As previously defined, each order coefficient a₀, a₁, a₂, b₁, or b₂ variates with the optical rotation angle γ, making the order coefficient a suitable optical feature for evaluating concentration of the bio-substance in the target area (A). Thus, the order coefficient set is output as the optical feature information, wherein the order coefficient set includes reflect light polarization feature and a target area scattering feature, which can be found in Table 1.

Optionally, as shown in FIG. 1A, the system (100) further comprises a pressure sensor (4) to detect a pressure applied on the target area (A) so as to produce a pressure information, and transmits the pressure information to the processor (3) for a compensatory process of the bio-substance concentration. Preferably, the pressure sensor (4) is a thin-film pressure sensor, such as an Arduino pressure sensing module, being signally connected to the processor (3), but not limited to this.

When the system (100) applied to the target area (A), maintaining a consistent condition during measurement of bio-substance level is required. In preferred embodiments, a pressure is applied when the system (100) contacts to the target area (A) such as a finger. Conceivably, as the target area (A) is subjected to pressure, conformational change of the epidermis or the dermis may disrupt the local bio-substance concentration.

For example, when diameter of the blood vessel is reduced in response to the pressure, blood flow volume changes, thereby resulting in fluctuation in blood sugar concentration in that target area (A). It goes without saying, such fluctuation requires some time to return to normal level. Namely, a stabilization time is required when pressure is applied to the target area (A).

In light of this, as shown in FIG. 2C, the concentration value determining step, Step S4, further comprises:
Step S40: combining a stabilization time information corresponding to the optical feature information, and an average concentration information to determine the concentration value; and
before the Step S40:
   Step S41: determining the stabilization time information based on a pressure information applied on the target area (A); and
   Step S42: determining the average concentration information by averaging two or multiple initial concentration values of the bio-substance detected within a period defined by the stabilization time information.

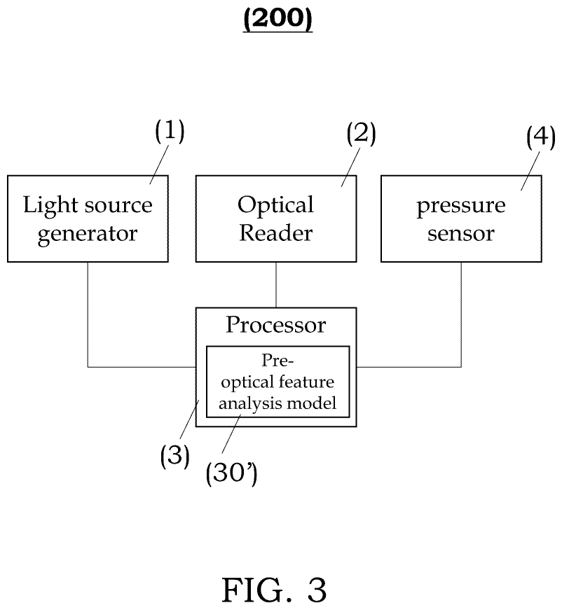

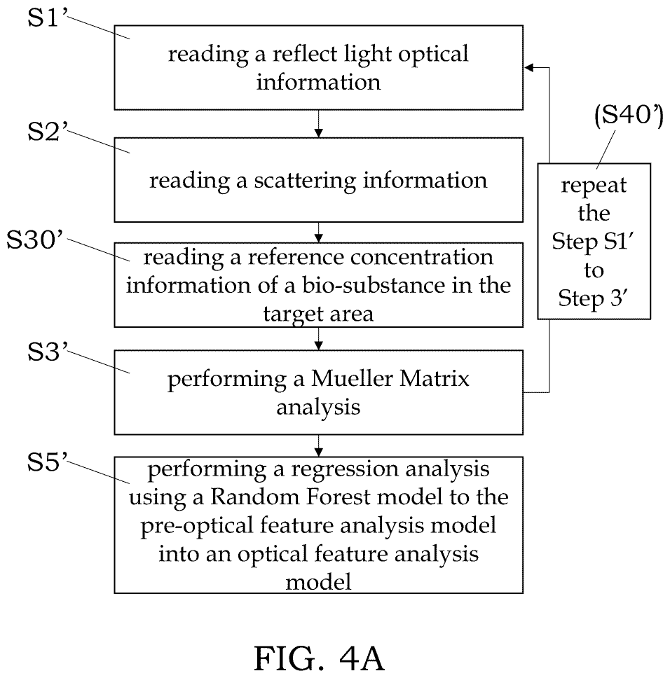

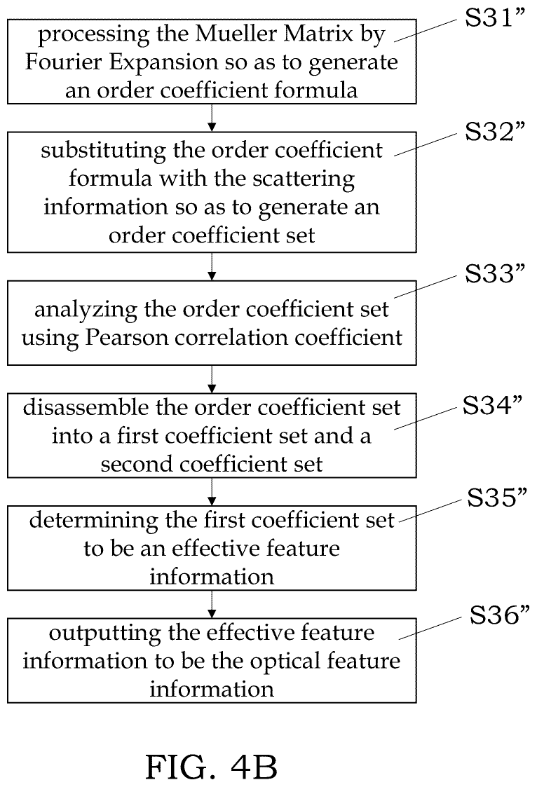

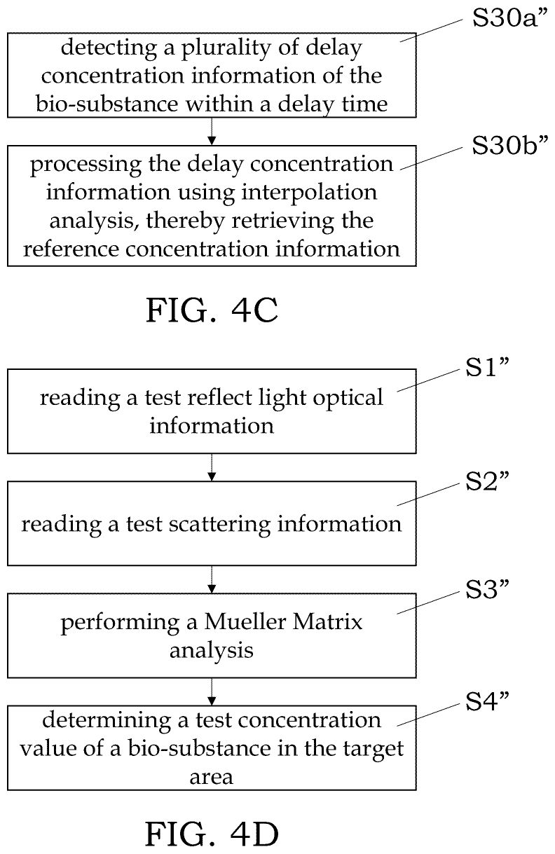

In another aspect of the present invention, as shown in FIG. 3, provides a system (200) for non-invasive optical measurement of bio-substances level in a biological tissue. The elements, connections between the elements and the working mechanism are overall the same as those in the first aspect of the present invention, but the system (200) comprises a pre-optical feature analysis model (30′) instead of the optical feature analysis model (30) in a training phase, wherein, as shown in FIG. 4A, the pre-optical feature analysis model (30′) to execute steps of:
Step S1′: reading a reflect light optical information corresponding to the reflect light, wherein the reflect light optical information comprises a reflect light intensity and a reflect light polarization information;
Step S2′: reading a scattering information corresponding to the target area (A), wherein the scattering information comprises one or multiple depolarization factors;
Step S30′: reading a reference concentration information of a bio-substance in the target area (A);
Step S3′: performing a Mueller Matrix analysis based on the reflect light intensity and reflect light polarization information so as to obtain an optical feature information, wherein the optical feature information comprises a reflect light polarization feature and target area scattering feature;
Step S40′: repeat the Step S1′ to Step 3′ to retrieve a plurality of the optical feature information and a plurality of the reference concentration information; and
Step S5′: based on the plurality of the optical feature information and the plurality of the reference concentration information, performing a regression analysis, based on a classification model, to train the pre-optical feature analysis model (30′) into an optical feature analysis model (30).

Optionally, the classification model may be a tree-based classification model, a binary classification, or a Support Vector Machine; the tree-based classification model may be Random Forest model, Gradient Boosting Machine or Extra-Randomized Trees model, but not limited to this; the binary classification model may be AdaBoost, but not limited to this.

In various embodiments, as shown in FIG. 4B, the Step S3′ distinguishes from the step S3 as the following steps are executed before obtaining the optical feature information:
Step S31′: processing the Mueller Matrix by Fourier Expansion so as to generate a pre-order coefficient formula corresponding to the reflect light intensity; and
Step S32′: substituting the pre-order coefficient formula with the scattering information so as to generate a pre-order coefficient set (Table 1);
Step S33′: analyzing the pre-order coefficient set using correlation analytic tool including Pearson correlation coefficient, Spearman correlation coefficient analysis, Euclidean distance analysis, or cosine similarity analysis, and Pearson correlation coefficient is preferred;
Step S34′: dividing the pre-order coefficient set into a first pre-coefficient set and a second pre-coefficient set, wherein a first correlation coefficient of the first pre-coefficient set is larger than a second correlation coefficient of the second pre-coefficient set; and Step S35′: determining the first pre-coefficient set to be an effective feature information;
Step S36′: outputting the effective feature information to be the optical feature information.

One skilled in the art may understand, correlation analysis requires at least two sets of pre-order coefficient. In the present invention, when using a correlation model to establish a correlation map, pre-order coefficient sets may be derived from the plurality of the optical feature information and the plurality of the reference concentration information, respectively. Correspondingly, these pre-order coefficient sets may be denoted as predicted order coefficient set and reference order coefficient set. Correlation analysis is performed with the predicted order coefficient set and the reference order coefficient set so as to retrieve correlation coefficient of each feature from the predicted order coefficient, and those predicted order coefficient set with higher correlation coefficients are selected as effective feature information.

Preferably, the pre-order coefficient set may be Fourier expansion coefficients as mentioned in the equation (3), and each pre-order coefficient of the pre-order coefficient set can be generated as the optical rotation angle γ variates. In one example, the bio-substance is blood glucose, and the rotation angle γ changes gradually from 23.9 to 23.18, with increments of 0.18, as the concentration of the blood glucose in the target area (A) varies. With the rotating polarizer (102a) rotating at the angular velocity from 0 to 360 degrees, the reflect light intensity and the reflect light polarization information can be obtained by the optical reader (2). The reflect light intensity and the reflect light polarization information may than be processed by Mueller Matrix analysis in combination with Fourier Expansion or fast Fourier Transformation to obtain the pre-order coefficient set including the reflect light polarization feature and the target area scattering feature. The pre-order coefficient set is then input into a classification model for regression training.

In some preferred embodiments, as shown in FIG. 4C, the reference concentration information is retrieved by a time delay processing, comprising:
Step S30′a: detecting a plurality of delay concentration information of the bio-substance within a delay time; and
Step S30′b: processing the delay concentration information using interpolation analysis, thereby retrieving the reference concentration information.

Apart from the pressure applied to the target area (A), other external factors at the target area (A) may cause fluctuation in the bio-substance concentrations. Taking skin surface of a fingertip for example, the placement and posture of the finger, skin oils, and laser intensity at the time contribute to these variations. A decreasing trend may be observed upon consecutive measurements on the target area (A), indicating that the initial values are causing the signal variations. Therefore, stable signals of the reflect light intensity may be observed upon consecutive measurements with a measurement interval, which allows the target area (A) to return to its initial state and reduce the overall trend. Exemplarily, the average values of the reflect light intensity obtained from the initial measurement interval and the ending measurement interval may be taken as the stable signal of the reflect light intensity, and thus subjected to Mueller Matrix analysis, thereby being output as optical feature information. The initial measurement interval may be 5 to 20 seconds since the system (200) starts to detects the reflect light, while the ending measurement interval may be 5 to 20 seconds before the system ends receiving the reflect light.

The plurality of the optical feature information and the plurality of the reference concentration information are then divided into training and validation subsets for cross-validation. With the prediction results being accurate and highly precise, for example, as the regression coefficient R² reaches over 0.99, the pre-optical feature analysis model (30′) is therefore validated as the optical feature analysis model (30).

In an inference phase, as shown in FIG. 4D, the optical feature analysis model executes steps of:
Step S1″: reading a test reflect light optical information corresponding to a test reflect light, wherein the test reflect light optical information comprises a test reflect light intensity and a test reflect light polarization information;
Step S2″: reading a test scattering information corresponding to a test target area, wherein the test scattering information comprises one or multiple test depolarization factors;
Step S3″: performing a test Mueller Matrix analysis based on the test reflect light optical information and test scattering information so as to obtain a test optical feature information, wherein the optical feature information comprises a test reflect light polarization feature and a test target area scattering feature; and
Step S4″: determining a test concentration value of a test bio-substance in the test target area according to the test optical feature information.

It should be noted that the Step S1″ to S4″ are basically the same as the Step S1 to Step S4 as described in the first aspect of the present invention. In view of the detailed description provided in the foregoing examples, further elaboration is deemed unnecessary.

The following examples are merely illustrative of the operational flow and technical efficacy of the present invention, and they are not intended to restrict the scope of the present invention.

### 1. Setup of Single Polarization Rotation System

The concept of a single-rotation polarizer system originates from the idea of Faraday rotation. Considering the expensive cost and challenges in miniaturization of the Faraday rotator, a rotating polarizer is adopted as an alternative for polarization states manipulation.

Shown in FIG. 1B is the schematic diagram of the system (100)/(200) in transmission mode.

On the other hand, shown in FIG. 1C is a modified device applying an aluminum coating to the rotating polarizer (2) within the rotating element, representing the system (100)/(200) in a reflection mode. This adaptation alters the light's trajectory from the transmissive mode to the reflection mode.

A 660 nm red laser beam passes through a 0-degree polarizer (101a), then through a rotating polarizer (102a) containing a 0-degree polarizer, which irradiates human skin or a tissue model. Scattered light is equipped with a 90-degree polarizer (101b) and is received by a light receiver. In the following examples, the 90-degree polarizer (101b) is a 90-degree polarizing mirror. During the measurement process, the rotating polarizer spins 360 degrees to capture signal changes.

### 1.1 the Transmission Mode

### 1.1.1 Simulation

First, set the basic parameters. The average intensity of the environment was 0.03, and the measurement duration for S0 (reflect light) was 8 seconds, with data collected every 0.005 seconds. The S0 intensity component includes random noise to simulate small fluctuations generated by the laser. The glucose concentration varied from 100 mg/dL to 500 mg/dL, with increments of 100 mg/dL, and the rotation angle γ changed gradually from 23.9 to 23.18 as the concentration varied, with increments of 0.18.

Commercial blood glucose meters served as control, and typically measure blood glucose levels within the range of 70 mg/dL to 600 mg/dL. Considering the blood glucose levels of diabetic patients may vary considerably due to factors such as diet, medication, and physical activity, the range of 100 mg/dL to 500 mg/dL can encompass the blood glucose levels of most individuals.

The optical rotation angle γ of the glucose may have a small random variation to simulate actual measurement conditions. The light absorption coefficient caused by glucose concentration changes very little for 660 nm red light, so the light absorption coefficient was set between 0.82 and 0.74 with an increment of 0.02.

The experiment involved a rotating polarizer (102a) inside the rotator spinning at a uniform angular velocity from 0 to 360 degrees, thereby obtaining rotating S0 signals and non-rotating S0 signals. The rotating S0 signals included reflect light optical information and scattering information. By performing a fast Fourier transform (FFT) on the rotating S0 signals, a spectrum was obtained. It can be found in Table 1 that the real part, denoted as A (the reflect light polarization feature), and the imaginary part, denoted as B (the target area scattering feature). The real part and the imaginary part were then input into a Random Forest model for regression training.

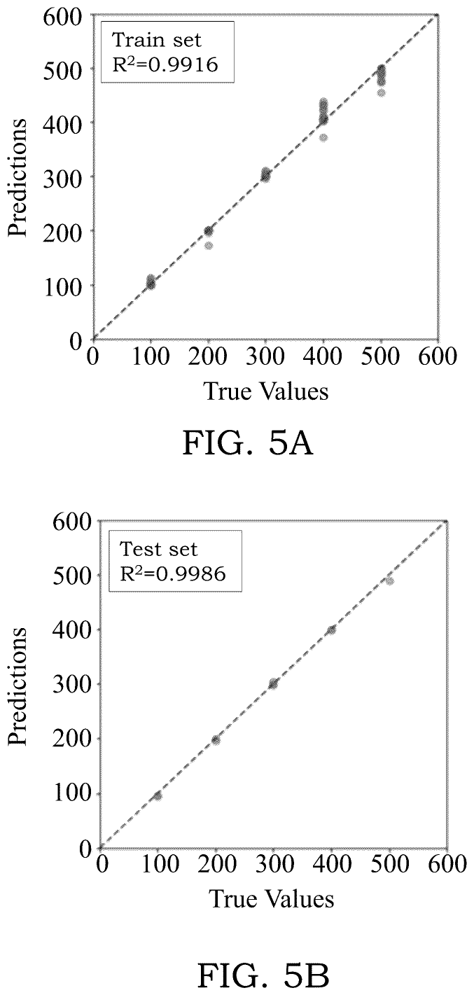

The sample data set was divided into a training set and a testing set at a ratio of 8:2. Within the training set, k-fold cross-validation was employed, dividing it into training and validation subsets in a 9:1 ratio, as shown in FIG. 5A. The prediction results are accurate and highly precise, as shown in FIG. 5B. With a regression coefficient R² of 0.9986, there is a very strong correlation.

### 1.1.2 Experimental Setup and Results in Phantom Tests

The actual setup for experiments: a laser generator (Cobolt 06-MLD 660 nm, 100 mW) was used as the light source generator; X-RSW60C-E03 (Zaber) was used as the rotating polarizer (102a); MODEL 2001-FS-M, New Focus) was used as the optical reader (2); the processor (3) included data acquisition system (DAQ) (NI 777960-01/Shielded BNC connector block with function generator BNC-2120) and software (Labview 2018) to run the optical feature analysis model (30); Arduino pressure sensor module was used as the pressure sensor (4).

After processing the experimental data, effective features were selected as input data for the Random Forest model. It is noted that effective features that exhibit high correlation with other features were removed based upon a feature correlation heatmap, and the importance of each feature or its correlation with the predicted glucose level was determined. So, exploring the correlation between each feature was the first step.

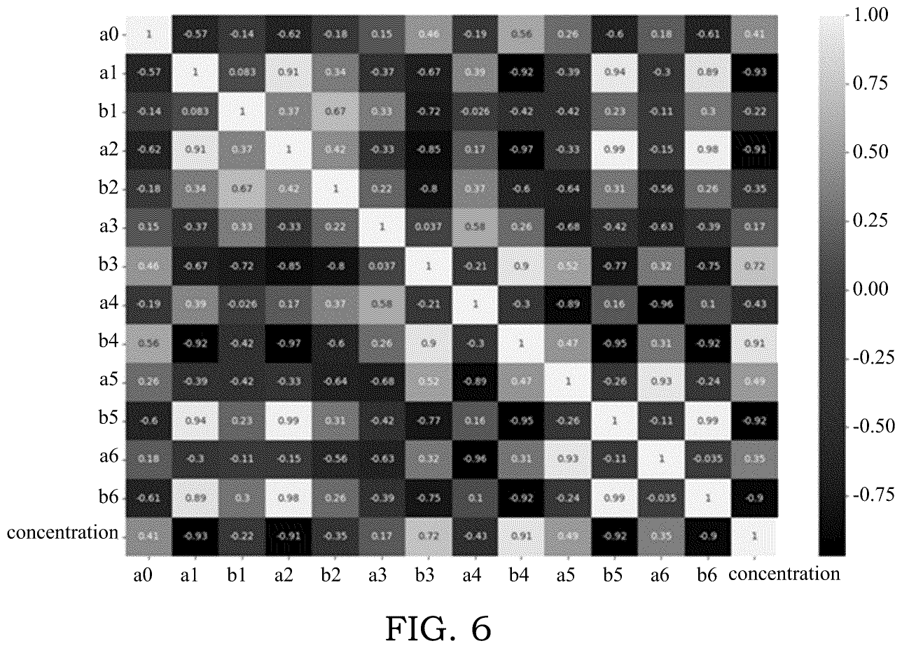

The heatmap is convenient to visualize the correlation, and the result of the heatmap is shown in FIG. 6. The order coefficients a₀, a₁, b₁, a₂, b₂, a₃, b₃, a₄, b₄, a₅, b₅, a₆, b₆ are the Fourier coefficients as defined in the first aspect of the present invention. The “concentration” refers to the glucose concentration of the phantom, namely the reference glucose concentration. It is noted that all the correlation coefficients between each feature were calculated using the Pearson correlation coefficient. The dark color means a high correlation between the two features, and the light color means a low correlation.

A mock simulating the scattering characteristics of human skin was created using a mixture of deionized water, glucose powder, and lipid particles. Five concentration ranges from 100 mg/dL to 500 mg/dL of a 2% lipid solution were prepared in 100 mg/dL increments to measure the relationship between glucose concentration and optical rotation angle. For each concentration, ten measurements were taken using rotating and non-rotating signals. The rotating signals underwent a fast Fourier transform (FFT) to obtain the corresponding spectra, and higher values were selected as one of the features for the Random Forest model.

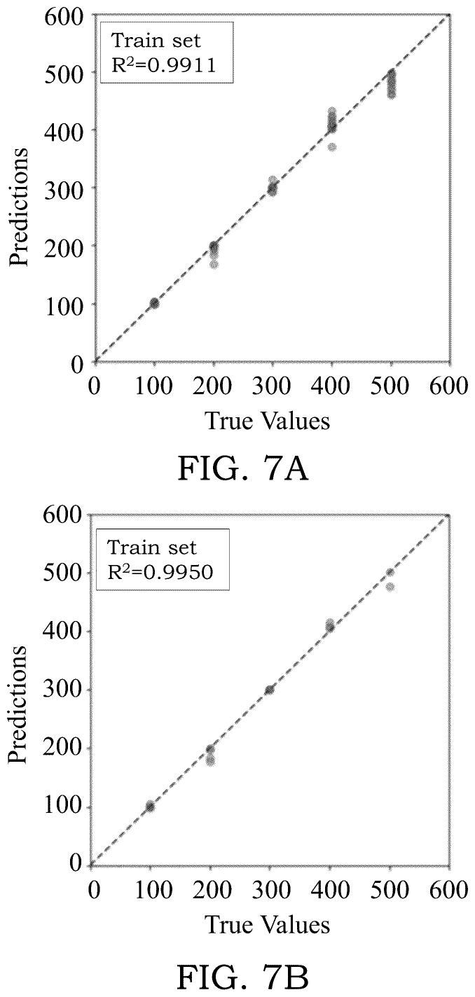

Next, these features, along with the non-rotating signals, were used to train the Random Forest model. The S0 signals of each concentration were used as the training set, with a training-to-prediction ratio of 8:2. Again, within the training set, k-fold cross-validation was employed, dividing it into training and validation subsets in a 9:1 ratio, as shown in FIG. 7A. FIG. 7B illustrates the results of the Random Forest training, showing high predictive accuracy. Additionally, the regression coefficient was found to be 0.9950.

### 1.2. Human Tests

### 1.2.1 Process in Human Tests

Human tests consist of three main stages for non-invasive measurement of glucose on the fingertip: pre-consumption, one-hour post-meal, and two and a half hours post-meal. During these intervals, participants also puncture their fingers to measure actual blood glucose levels for comparison. When the finger was placed on the holder, a consistent position and uniform pressure needed to be carefully maintained. Subsequently, a 660 nm laser was launched into the finger and then a LabVIEW-based DAQ control program was to proceed with capturing the signals reflected from the finger. Each measurement involves five consecutive readings to ensure accuracy. After completing measurements in the three stages, the data were analyzed.

### 1.2.2 Pressure Effect for Human Tests

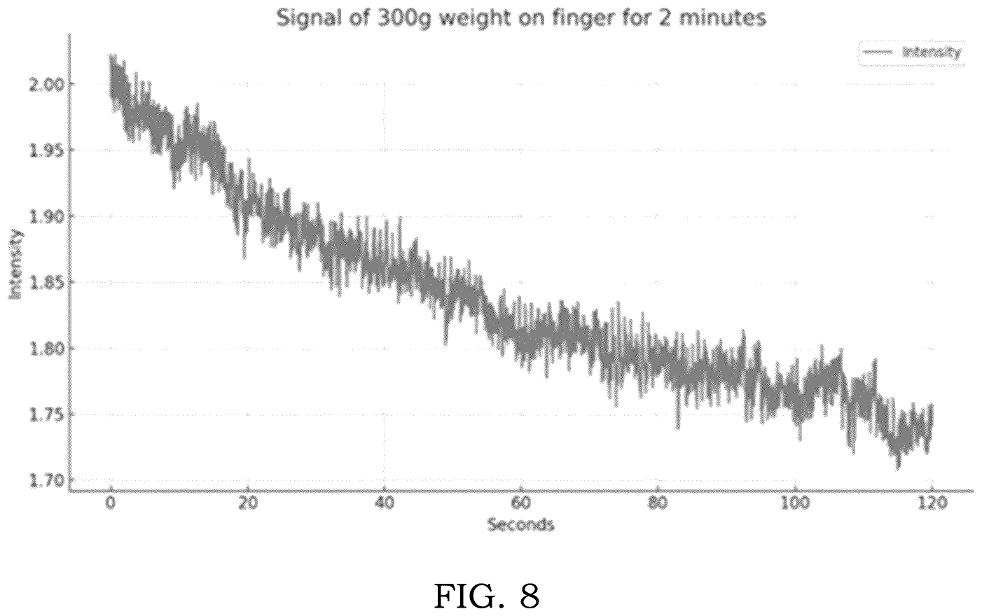

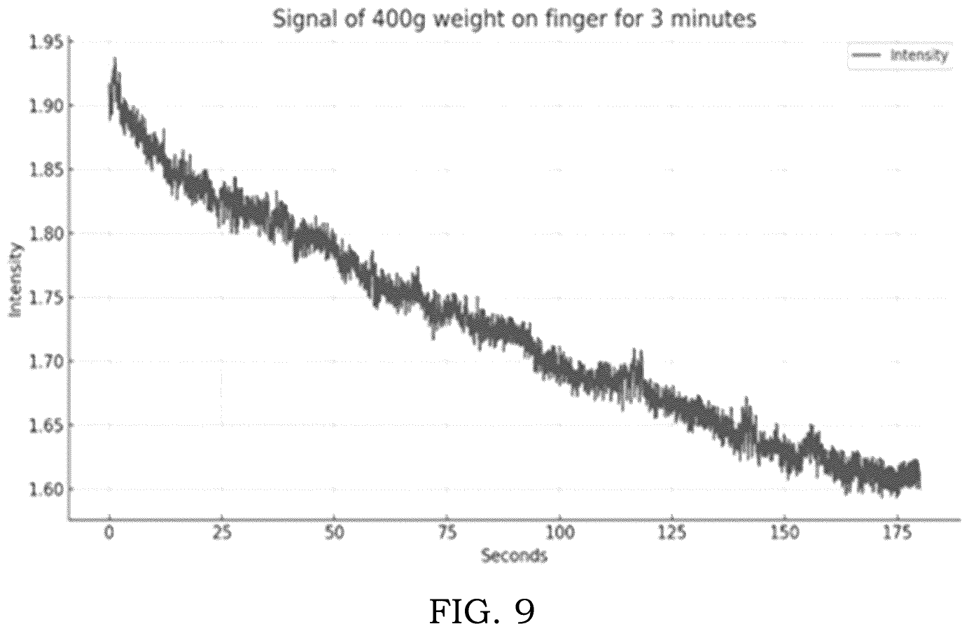

In order to maintain consistent finger conditions during each measurement, pressure is applied to the finger. When the skin is subjected to pressure, it undergoes changes that require some time to stabilize. FIGS. 8 and 9 illustrate the signals obtained at the same blood glucose level with different levels of pressure applied to the finger while the rotary remains stationary. FIG. 8 corresponds to data collected with a 300-gram weight applied for 2 minutes, while FIG. 9 corresponds to data collected with a 400-gram weight applied for 3 minutes. The sampling frequency is 500 samples per second, resulting in 60,000 data points for FIG. 8 and 90,000 data points for FIG. 9. It can be observed that the greater the force applied to the finger, the longer the required stabilization time. Therefore, in the human experiments, an Arduino pressure sensing module was installed as a feature value for Random Forest analysis.

### 1.2.3 Initial Value Influence

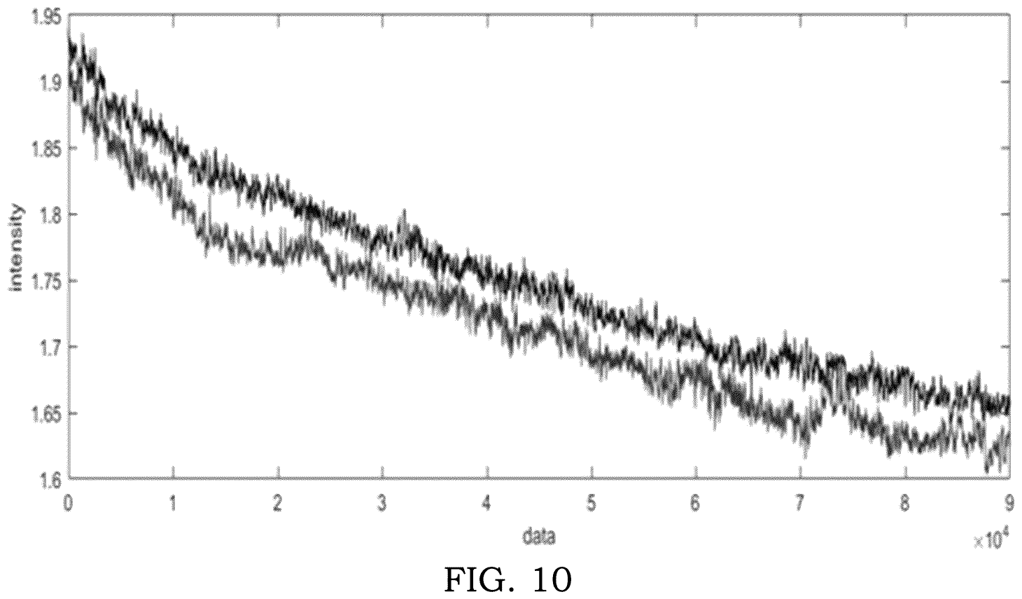

Referring to FIG. 10, setting measurement interval of 1 minute allows the skin to return to its initial state and reduce the overall trend. The similarity in the decreasing trend indicates that the initial values are causing the signal variations. The initial intensity values were obtained immediately after applying pressure to the finger; thus, they were not influenced by changes in the skin's internal state caused by pressure. Consequently, external factors such as the placement and posture of the finger, skin oils, and laser intensity at the time may contribute to these variations. Therefore, the average of the first 10 seconds and the average value of the last 10 seconds of the stable signal's intensity are taken as feature points.

### 1.2.4 Effective Feature Selection

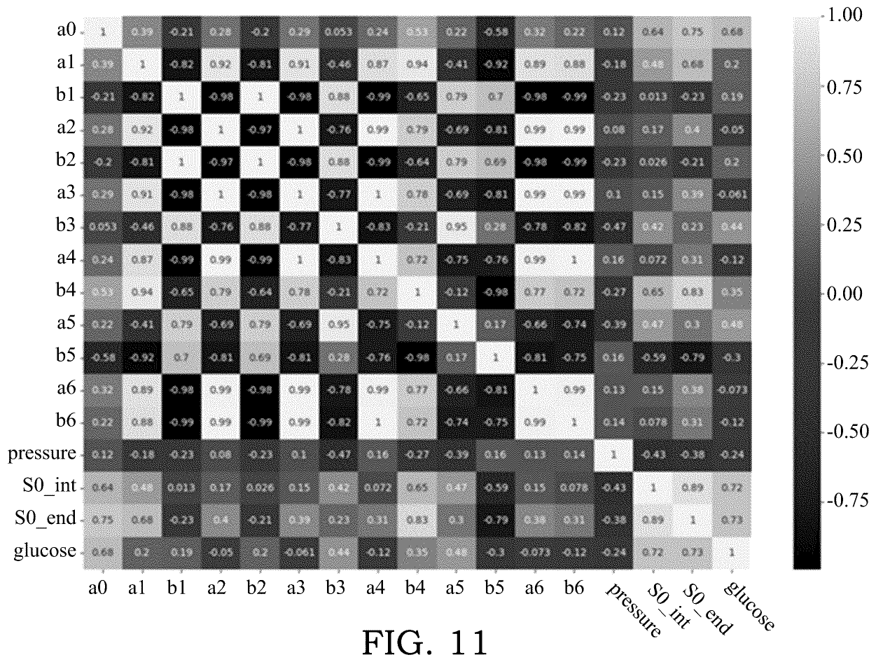

The correlation between features and blood glucose levels is crucial in the optical feature analysis model (30). High collinearity among features can reduce the accuracy of Random Forest model predictions. Therefore, removing highly collinear features can prevent a decrease in the accuracy of machine learning predictions. In Table 1, there is a significant correlation (collinearity) between a₀, a₁ and a₂, as they are all related to the parameter γ. However, in the human experiments depicted in FIG. 11, Fourier expansion coefficients a₀, a₁, b₁, a₂, b₂, a₃, b₃, a₄, b₄, a₅, b₅, a₆, b₆ are the Fourier coefficients as defined in the first aspect of the present invention. In FIG. 11, “Pressure” refers to the pressure means, while “s0 int” and “s0 end” denote the average of the first 10 seconds and the average value of the last 10 seconds of the stable signal's intensity, respectively.

The collinearity between a₀ and glucose is relatively high, with a correlation coefficient of 0.6779, whereas the correlation between a₁ and glucose level and that between a₂ and glucose level are still lower, with correlation coefficients of 0.2 and −0.05, respectively. This unexpected in correlation with glucose levels in a₁ and a₂ may be attributed to the opposing effects of proteins and glucose on the optical rotation angle of light in the visible light spectrum within the human body. These opposing effects tend to cancel out each other to some extent, leading to a reduced correlation with glucose levels in a₁ and a₂. Additionally, as the frequency increases, their amplitudes decrease, indicating a reduction in scattering. This also explains why the reduced correlation with glucose levels in a₁ and a₂ is relatively low. Furthermore, it is confirmed that a₀ is directly related to the DC term, which responds to not only the rotation angle but also the scattering issue. Thus, a₀ still has the strongest correlation with glucose level amounts a₁ and a₂. This phenomenon suggests that the interaction of light with proteins and glucose in the human body is complex and can vary based on the specific wavelengths and frequency components involved.

Alternatively, in the phantom experiment, as there were no interfering substances such as albumin or globulin used in the formulation, in FIG. 9, the effects of a₀, a₁ and a₂ on “γ” were not interfered with by a specific protein to glucose level. As a result, the DC component is predominantly influenced by the parameters “γ” and scattering, while the AC component weakens as the frequency domain increases, indicating reduced scattering. This phenomenon can be attributed to the absence of interfering substances in the phantom experiment, allowing the parameters “γ” and scattering to have a more pronounced effect on the DC and AC components of a₀, a₁ and a₂. Consequently, a₁ and a₂ exhibit a stronger correlation with glucose concentration when compared to a₀.

### 1.3 Results of Human Tests in Compensating Time Delay

1.3.1 Tests without Compensating Time Delay

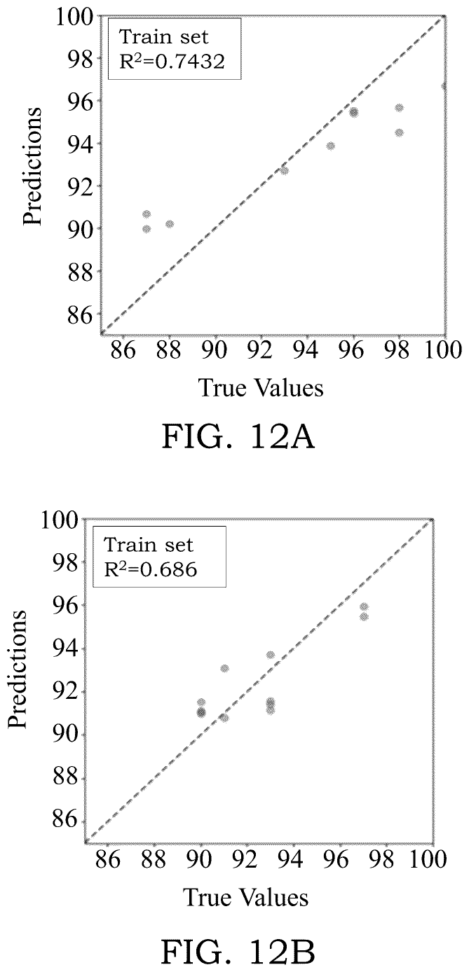

Over a span of three consecutive days, human subjects were monitored with blood glucose levels measured three times each day and three samples taken during each measurement. As a result, a total of 27 data points were generated during the experimental process. Based on FIG. 10, the final effective features, such as signals at the Fourier expansion coefficients a₀, a₁, as, b₁, b₂, b₃, and b₅, the pressure mean, and the average value of the last 10 seconds of the stable signal's intensity (s0 end) shown in FIG. 11, were chosen as input data for the Random Forest model for regression training. Due to variations in the placement of fingers during human measurement, inherent measurement errors were present. To mitigate this, all the data underwent Random Forest prediction and analysis, allowing for the exclusion of data with significant predictive inaccuracies. FIG. 12 shows the results of the Random Forest analysis, where all data was used for training and prediction. Similarly, the training set and test set are divided into 8:2. Within the training set, k-fold cross-validation was employed, dividing it into training and validation subsets in a 9:1 ratio, as shown in FIG. 12A. The result in FIG. 12B is not very good because of the time delay caused by the time difference.

### 1.3.2 Compensated Results for Time Delay

Under 660 nm red light, the sensing depth of diffuse reflection is relatively shallow, and it cannot reach the dermis layer within micro vessels. As a result, it can only sense the interstitial fluid within the epidermis. The interstitial fluid transports substances through diffusion, a process that takes time and leads to delayed fluctuations in glucose levels in the interstitial fluid. In comparison to blood, there's a delay in the concentration of glucose in the interstitial fluid. This delay arises because the diffusion of glucose requires a certain amount of time and is not instantaneous. Therefore, compensation is necessary to enhance prediction accuracy. The compensation method employed in this section is interpolation.

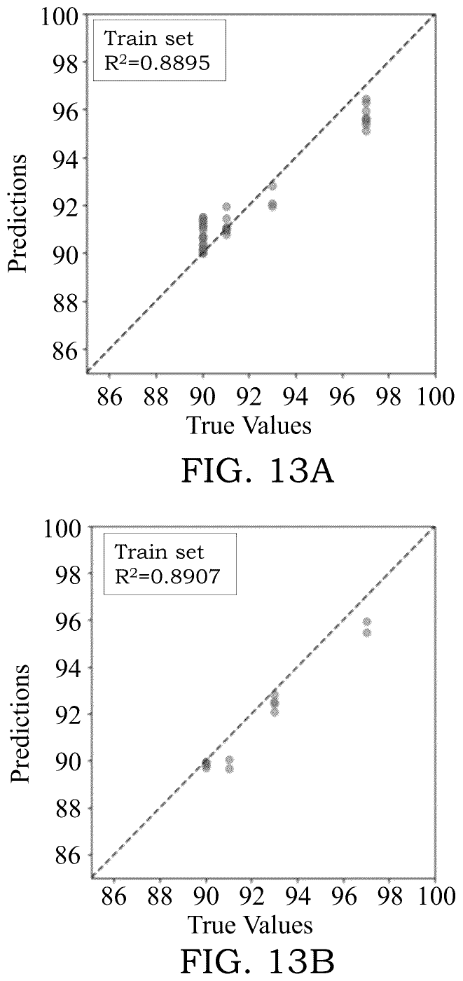

The delay times for glucose increase and decrease are different, and the compensation time varies from person to person. Thus, the addition or reduction of compensation codes has its own random parameters. Hereinafter, a baseline delay time set at 17 minutes. Subsequently, all the data is used for Random Forest model for regression training. FIGS. 13A to 13B show the Random Forest training and prediction results for all filtered profiles after compensation.

Again, the data was divided in a ratio of 8:2 for training and prediction. Within the training set, k-fold cross-validation was employed, dividing it into training and validation subsets in a 9:1 ratio, as shown in FIG. 13A. The results shown in FIG. 13B indicate a regression coefficient R² of 0.8907 and a MARD of 6.8%. It is evident that after compensation, the prediction accuracy improves further. It is noted that the MARD is a critical metric used to assess the accuracy of glucose measurement systems. It calculates the average of the absolute differences between predicted and actual glucose values, relative to the actual values. A lower MARD indicates higher accuracy, essential for reliable glucose monitoring. The international standard for MARD in glucose meters is generally considered to be below 10%.

### 1.4. Results of Institutional Review Board (IRB) at NCKU Hospital

As for real world validation, the research protocol had been submitted to and is currently under consideration by the IRB (Code No. A-ER-112-025). Two participants diagnosed with diabetes and two healthy counterparts were involved, with a planned duration of three months. The proposed schedule includes measurements conducted twice a week, totaling 24 days. Daily measurements included pre-meal, 1-hour post-meal, and 2.5-hour post-meal intervals. The measurement process incorporated fingerstick blood glucose tests, resulting in 72 measurements per individual. This comprehensive approach aimed to capture a detailed and varied dataset, enabling a thorough analysis of glucose level variations in different contexts. It is noted that when the finger was placed on the holder, a consistent position and uniform pressure needed to be carefully maintained. Subsequently, a 660 nm laser was launched into the finger, and then a LabVIEW-based DAQ control program proceeded with capturing the signals reflected from the finger. Each measurement involved five consecutive readings to ensure accuracy. After completing measurements in the three stages, the data was analyzed individually for four participants.

### 1.4.1 Imbalanced Data

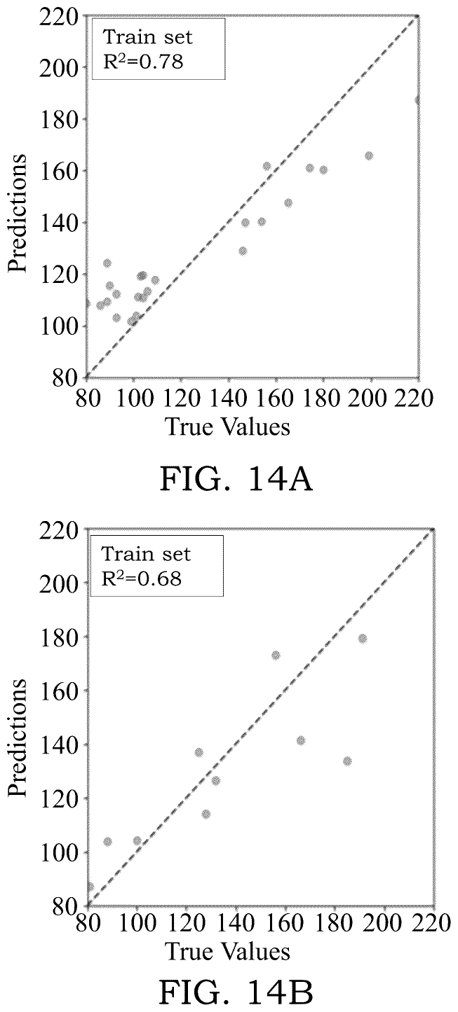

Data imbalance can result from biases in the sampling process, such as biased sampling methods or measurement errors. It may also stem from inherent characteristics of the problem domain. For instance, the IRB human measurements demonstrate that fluctuations in blood glucose levels before and after meals in diabetic subjects typically lead to data that is skewed toward either low or high glucose levels, with limited data in the intermediate range. FIGS. 14A to 14B illustrate how this skewed distribution is not ideal for machine learning, as it may cause model outputs to deviate, adversely affecting performance and making it challenging to accurately predict mid-range blood glucose levels. FIG. 14A shows the distribution of the training set, highlighting the issue of data imbalance. Conversely, the test set depicted on FIG. 14B achieved the R² of only 0.68, further demonstrating the negative impact of data imbalance on model performance.

### 1.4.2 Weight Adjustment and Random Over Sampling

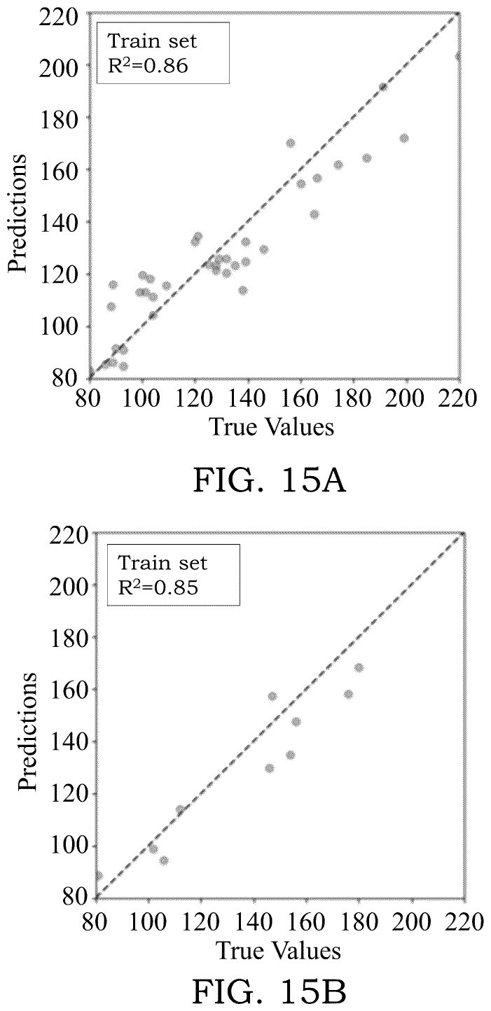

In regression, weight adjustment involves assigning different weights to observations based on specific criteria. These weights help address data imbalance issues. The purpose of weight adjustment is to make the model focus more on minority samples, thereby improving prediction accuracy for these samples. By assigning appropriate weights, the regression model can prioritize certain observations, thus better addressing specific problems in the data. The principle of random over-sampling is to balance the dataset's distribution by increasing the number of less frequent samples. Randomly selecting and duplicating these samples can enhance the model's performance in handling imbalanced data. The combination of random over-sampling in the preliminary stage and subsequent weight adjustment significantly improved the model's performance. As illustrated in FIGS. 15A to 5B, FIG. 15A shows an increased number of blood glucose values in the range of approximately 120˜160 mg/dL compared to FIG. 14A, while FIG. 14B shows an improvement in the R² to 0.85.

### 1.4.3 IRB Measurement Results

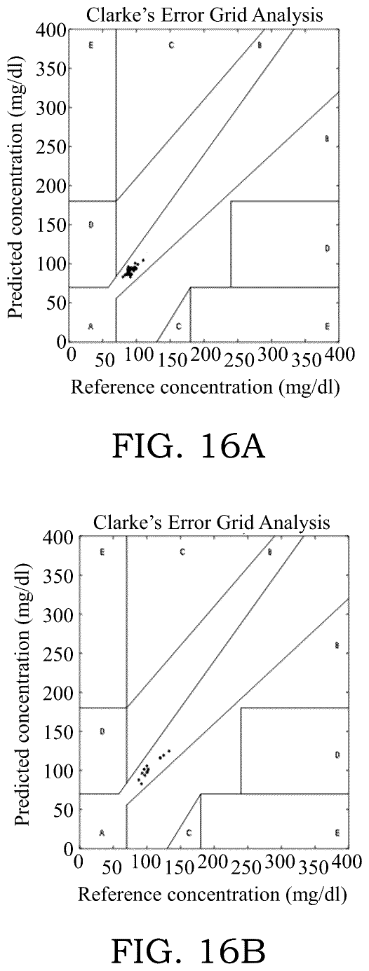

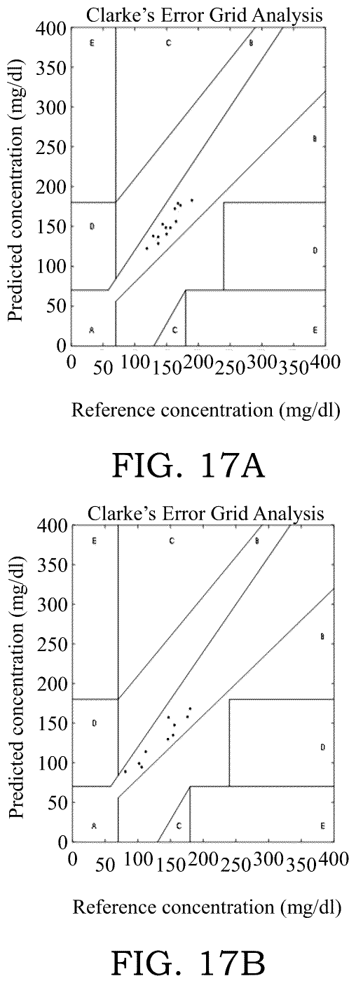

FIG. 16A represents data from healthy individual 1 while FIG. 16B corresponds to healthy individual 2. In another aspect, FIG. 17A pertains to data from diabetic patient 1, while FIG. 17B represents diabetic patient 2. It is evident from the graphs that, particularly at actual blood glucose concentrations below 150 mg/dl, most data points closely match the reference concentrations, indicating that the blood glucose monitoring device performs with high accuracy, as detailed in Table 2. As the reference glucose concentrations increase, there is a slight upward shift in the data points, suggesting a potential decrease in the device's accuracy at higher glucose levels, although this deviation remains within an acceptable margin. Notably, there are no data points falling within Zones C, D, and E, indicating an absence of severe measurement errors.

TABLE 2

| | healthy | healthy | diabetic | diabetic |
|---|---|---|---|---|
| | individual 1 | individual 2 | patient 1 | patient 1 |
| MARD | 1.535% | 3.88% | 4.44% | 7.79% |
| R² | 0.856 | 0.864 | 0.836 | 0.855 |

Due to complicated factors in extracting the glucose level from the finger holder and surrounding noise, the methodology proposed in this study is based upon domain knowledge and data science. Therefore, the effective features analyzed by a correlation matrix as input datasets for the Random Forest model are needed before proceeding with the training for predictions. It is noted that the stable signal data from the initial and final signal trail in 10 seconds are two effective features aiming to improve the handling of external factors to finger. Furthermore, an Arduino thin-film pressure sensor was added as an additional feature for the Random Forest model. Finally, there are signals at the Fourier expansion coefficients: a₀, a₁, a₅, b₁, b₂, b₃, and b₅, the pressure means, and the average value of the last 10 seconds of the stable signal's intensity (s0 end) selected as effective features. Finally, an interpolation method was employed to compensate for the delayed interstitial fluid glucose concentration. This resulted in an increase in the regression coefficient to 0.8907 and a decrease in MARD to 6.8% for healthy participants in the lab, further improving prediction accuracy. In addition, IRB measurement results indicate that the monitoring device exhibits high accuracy within the blood glucose concentration range below 150 mg/dL. Although accuracy slightly decreases at higher glucose levels, the deviation remains within an acceptable range. Importantly, no measurement results fall within the severe error zones (Zones C, D, and E), demonstrating the device's reliability in measuring blood glucose levels across the entire test range. It is noted that the Random Forest training model for the signal from a single polarization rotator system in extracting the glucose concentration is only for an individual and not for everyone as an invasive device; therefore, it is more like a custom-made device in this study.

It is noted that techniques involving polarized light can reveal anisotropic properties of biological tissues, such as birefringence and dichroism, which are often associated with pathological changes. For instance, polarized light imaging is used to detect alterations in collagen structures, relevant for diagnosing conditions like cancer and fibrosis. Additionally, polarized light is sensitive to glucose concentration due to the optical activity of glucose molecules, causing measurable rotation in the plane of polarization. Therefore, more features from domain knowledge integrated with data science could be an efficient method to enhance the sensitivity of non-invasive measurement of glucose.

### 2. Reflection Mode

### 2.1 Simulation Results

The specific rotation angle of glucose was set approximately 45.23°·mL/dmg at a wavelength of 660 nm. To simulate glucose concentrations ranging from 100 to 500 mg/dL in increments of 100 mg/dL, the rotation angle γ is adjusted from 0.18° to 0.9° in steps of 0.18°. Additionally, intensity noise is incorporated into the simulation to mimic real-world conditions, with the input intensity following a random distribution of 5.18±0.001, representing experimental noise. The angle of the rotating polarizer is varied from 0° to 360° in increments of 0.1°, simulating a complete revolution of the polarizer. Five distinct values of γ are calculated separately for each rotation angle.

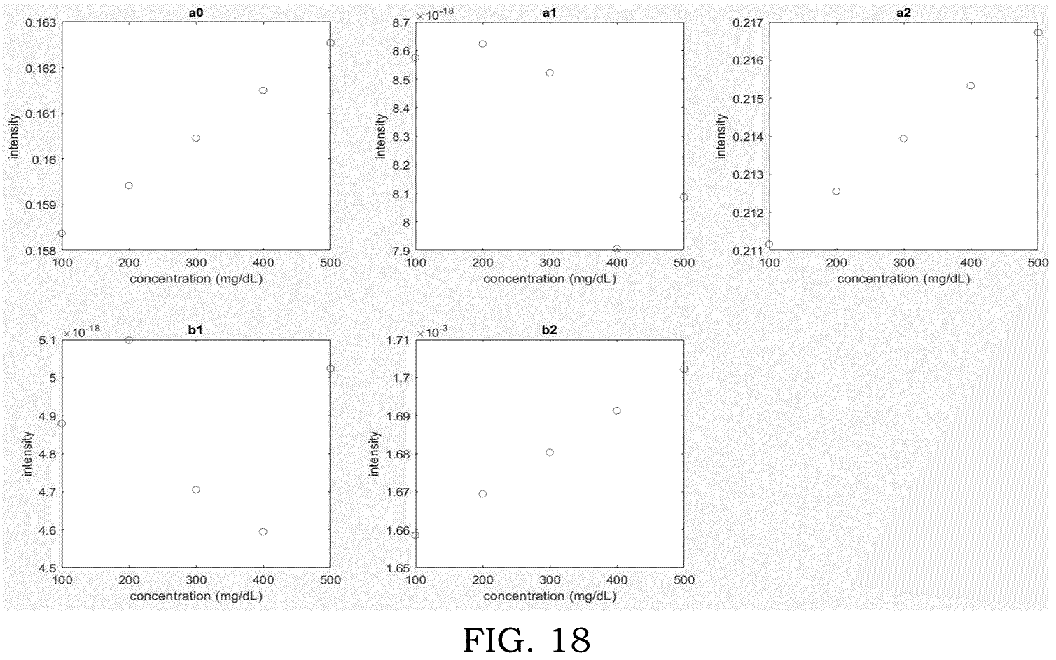

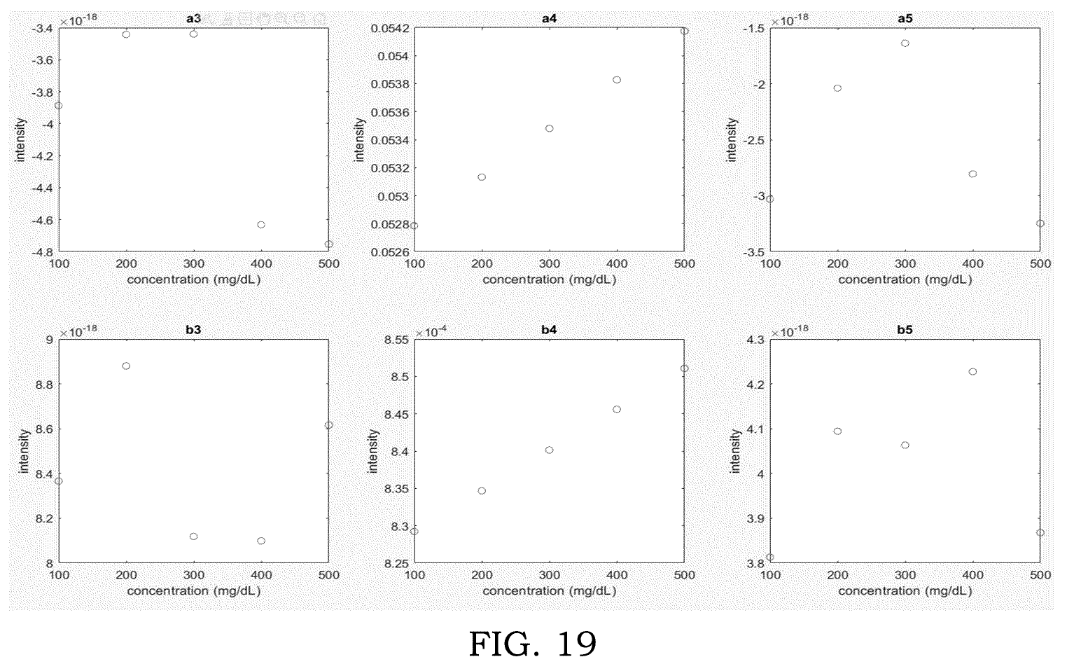

To overcome errors in the actual experimental value of γ, the simulation was carried out ten times for each γ to capture the variability of each feature. Following this, the corresponding intensities were obtained. Fast Fourier Transform (FFT) was used to convert the time-domain intensity signals into the frequency domain. After the FFT process, the Fourier Expansion coefficients were identified. The outcomes of the coefficients are presented in FIG. 18 and FIG. 19, where real part is denoted as “a”, and imaginary part is denoted as “b”.

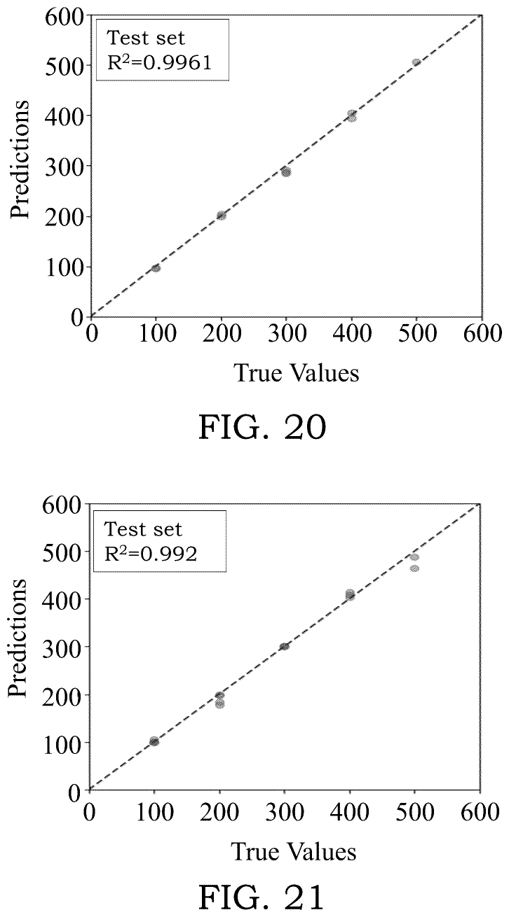

The Fourier Expansion coefficients were fed into a Random Forest training model, with the dataset of 50 samples split into training and test sets at an 8:2 ratio. The results of the prediction were accurate and demonstrate high precision, as illustrated in FIG. 20. With a regression coefficient of 0.996 and the MARD was 1.95%, there is a very strong correlation. As compared to the transmission mode, introducing intensity noise into the simulation enhances the model's robustness and applicability to real-world conditions, resulting in more observed frequency domain responses under the same intensity level. Additionally, the integration of machine learning techniques, specifically employing a Random Forest training model, improves prediction accuracy.

### 2.2 Human Tests for Verifications

For human experiments, the equipment mentioned in section 1.1.2. The improved device uses an aluminum-coated polarizer on the rotator. Aluminum was chosen for its high reflectivity, particularly in the visible spectrum, including the 660 nm red laser light used in this example. This coating ensures that most of the laser light is reflected toward the target area (A), such as human skin or tissue models. This increases the signal strength received by the detector. Additionally, the aluminum coating helps calibrate the machine more accurately, ensuring consistent and reliable measurement results.

The phantom formulation is shown in Section 1.1.2. For each concentration level, measurements were conducted ten times using both rotating and non-rotating signals. The rotating signals underwent Fast Fourier Transform (FFT) to acquire the respective spectra, which were subsequently chosen as features for machine learning based on the methodology described in section 1.2.4. Subsequently, the dataset was divided into an 8:2 training-to-prediction ratio and input into the model for training, yielding an R² value of 0.992 and the MARD was 2.358%, as shown in FIG. 21. This result also verified the simulation results.

### 2.3 Experimental Results of Human Tests

After the data preprocessing and feature selection mentioned in section 1.2.4, human participants were continuously monitored for one week, with 15 measurements taken at three different time periods, accumulating a total of 45 data points. After the experiment concluded, data underwent preprocessing, feature selection, and other actions following the methodology outlined in the previous section 1.2.4. The data was then split into a training set (80%) and a test set (20%) and fed into a random forest machine learning model for training.

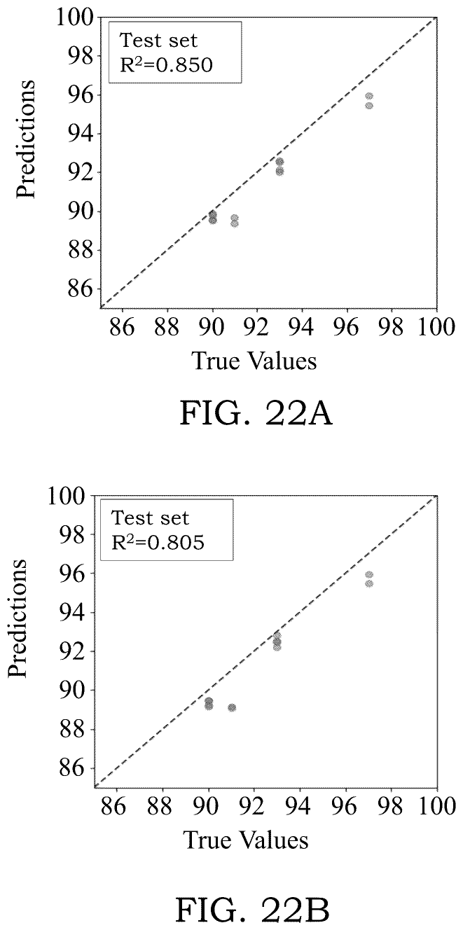

To validate the use of Stepwise Regression, the top 5 features were selected. The highly correlated features were excluded so that model complexity and multicollinearity could be avoided. Then, stepwise regression was employed to select the final top 5 features, including FFT coefficients a₁, b₅, b₆, data from the pressure sensing module, and the averaged signal intensity from the first 10 stable seconds. FIG. 22A shows the prediction results of the pre-optical feature analysis model (30) using the Stepwise Regression method, and, instead, FIG. 22B shows the prediction results without using the Stepwise Regression method.

The Stepwise Regression method iteratively adds or removes variables to balance pre-optical feature analysis model (30′) accuracy and simplicity. Starting with pre-optical feature analysis model (30′) in a simplified version, variables were adjusted based on statistical criteria to improve the model (30′). Finally, the pre-optical feature analysis model (30′) was improved to be a workable optical feature analysis model (30) being concise and effective, containing key features for predicting the target variable. This approach enhances interpretability and predictive accuracy while preventing overfitting.

### 2.4 Verifications of IRB Databank

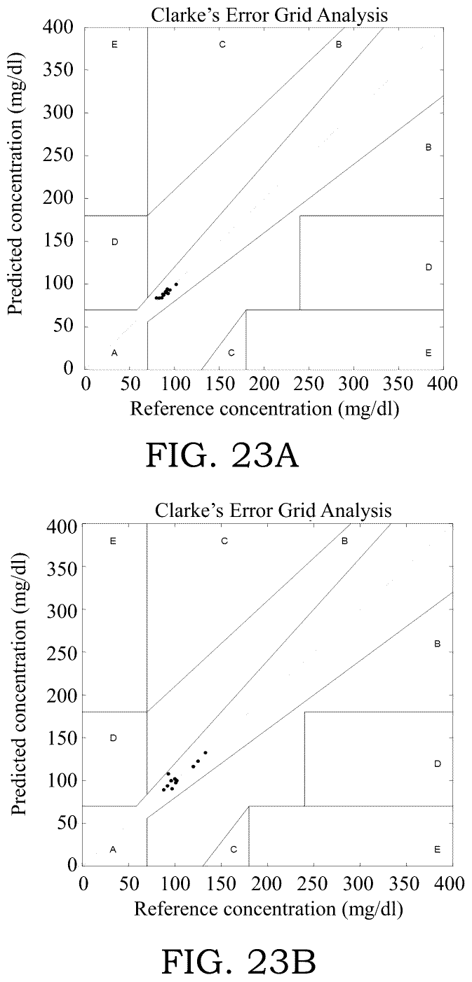

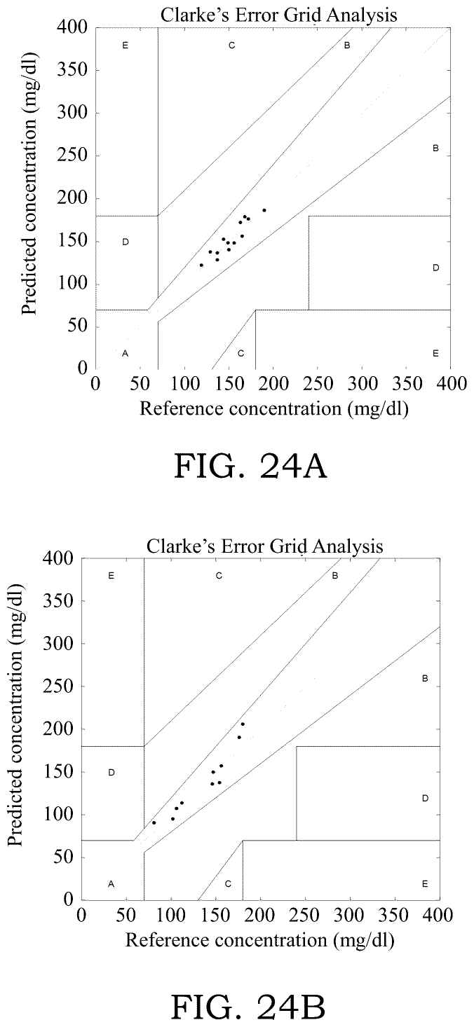

According to the Stepwise Regression method, the results of the human trials yielded an R² of 0.85. To validate this methodology, Institutional Review Board (IRB) experimental data were employed as mentioned in section 1.4, and the data were subjected to identical processing. The results of healthy participant 1 and healthy participant 2 are presented respectively in FIGS. 23A and 23B, while the results of diabetic participant 1 and diabetic participant 2 are presented in FIGS. 24A and 24B. Table 3 lists the MARD and R² for each subject, with the outcomes being quite remarkable and validating the feasibility of this method. It proved in section 1.2.4 that the manually selected 8 effective features, as shown in Table 4. Subsequently, the Stepwise Regression method further narrowed down these 8 effective features to 5, resulting in optimal model performance.

TABLE 3

| | healthy | healthy | diabetic | diabetic |
|---|---|---|---|---|
| | participant 1 | participant 2 | participant 1 | participant 2 |
| MARD | 1.81% | 3.61% | 4.29% | 6.46% |
| R² | 0.853 | 0.858 | 0.845 | 0.861 |

TABLE 4

| healthy | healthy | diabetic | diabetic |
|---|---|---|---|
| participant 1 | participant 2 | participant 1 | participant 2 |
| a1 | a1 | a3 | a1 |
| b1 | b01 | a4 | a3 |
| a3 | a3 | b4 | b3 |
| a4 | a4 | a5 | a4 |
| b5 | a5 | a6 | b4 |
| a6 | b5 | b6 | b6 |
| Pressure | b6 | Pressure | Pressure |
| S0_end | Pressure | S0_initial | S0_initial |

The final selection of features varied for each participant, as depicted in Table 5. The features in Table 5 exhibit FFT coefficients having higher correlation with glucose concentration, as observed in FIGS. 12A to 12B. Some features, despite showing lower correlation with glucose concentration in FIGS. 12A to 12B, were still selected as input feature values. This may stem from the consideration of inter-feature collinearity during the modeling process.

TABLE 5

| healthy | healthy | diabetic | diabetic |
|---|---|---|---|
| participant 1 | participant 2 | participant 1 | participant 2 |
| a1 | a1 | a3 | a1 |
| a3 | a4 | a4 | a3 |
| a4 | b5 | a5 | b6 |
| b5 | b6 | a6 | Pressure |
| Pressure | Pressure | b6 | S0_initial |

Collinearity refers to the scenario where features exhibit a high degree of correlation with each other. This can lead to instability in the model, resulting in decreased predictive performance on new data. To address this issue, feature selection or dimensionality reduction techniques are commonly employed to reduce the dimensionality of the feature space. In such cases, even if certain features display low correlation with glucose individually, they are still chosen as input feature values due to their high in-correlation with other features. Retaining these features helps mitigate collinearity, thereby enhancing the stability and generalization capability of the model. Thus, while these features may not exhibit high correlation with the target variable in isolated analyses, their inclusion can still benefit the overall model performance. This also suggests a shift towards customization in future commercialization strategies.

As shown in Table 5, variations in individual finger attributes, such as thickness affecting pressure values and the position of laser light impacting initial signal values, underscore the need for tailored approaches. Nonetheless, FFT coefficients dominate most features. Overall, this underscores the necessity of considering in-correlation among features, even if their correlation with the target variable is low, to ensure robust model performance.

### 3. Beneficial Effects of the Present Invention

The present invention solves issues of light signal instability due to complicated factors in extracting the glucose level from the finger holder and surrounding noise. By selecting the effective features analyzed by a correlation matrix as input datasets for the Random Forest model, the stable signal data from the initial and final signal trail in 10 seconds are two effective features facilitating to minimize the effect of external factors to finger.

Furthermore, Arduino thin-film pressure sensor is also applied to provide pressure information as an additional feature for the Random Forest model. The Fourier expansion coefficients: a₀, a₁, as, b₁, b₂, b₃ and b₅, the pressure means, and the average value of the last 10 seconds of the stable signal's intensity (s0 end) are selected as effective features.

An interpolation method was also employed to compensate for the delayed interstitial fluid glucose concentration. This resulted in an increase in the regression coefficient to 0.8907 and a decrease in MARD to 6.8% for healthy participants in the lab, further improving prediction accuracy. In addition, in real-world validation, IRB measurement results indicate that the monitoring device exhibits high accuracy within the blood glucose concentration range below 150 mg/dL, with subtly reduced accuracy at higher glucose levels.

Significantly, no measurement results fall within the severe error zones (Zones C, D, and E) when Clarke Error Grid analysis was applied, demonstrating the device's reliability in measuring blood glucose levels across the entire test range. By using the Random Forest training model for the optical feature information retrieved from a single polarization rotator system, the predicted glucose level can be output with accuracy, and thus the single polarization rotator system may serve an ideal option as a custom-made system for bio-substance level in a biological tissue.

As proteins and glucose influence each other's optical rotation angles within the human body, whereas their effects are less pronounced in the non-visible light spectrum. Based on the experimental results in phantom and human tests, it may be inferred that substances influencing mutual interactions within the non-visible light spectrum have a smaller impact, potentially reducing interference between different bio-substances.

Apart from that, to address the time delay effect, Stepwise Regression is also employed to select the top features for predicting glucose concentration. With compensation of time delay significantly enhances model accuracy, as shown by comparing raw data with and without compensation. The optical feature analysis model (30) was also validated by Human trials, yielding an R² of 0.85, and further refined the features from 8 to 5 for optimal model performance.

By integrating these technical solutions, the present invention significantly enhanced the single-polarization rotation system in both transmission mode and reflection mode for non-invasive glucose level monitoring. Analysis based on optical rotation angles and light intensity signals, with incorporation of pressure effects and time delay compensation for effective feature selection, allows a high-accuracy random forest model to be developed. The model has also been validated through phantom model experiments and human trials, bolstering the predictive model's reliability and suitability for clinical application.

What is claimed is:

1. A system for non-invasive optical measurement of bio-substance in a biological tissue, comprising:
a light source generator, configured to generate an incident light to irradiate at a target area on an individual;
an incident polarizer set, positioned on an incident light optical path of the incident light, and being configured of a 0-degree polarizer and a rotating polarizer in a sequential manner, wherein:
the rotating polarizer spins about the incident light optical path at an angular velocity; or
the rotating polarizer spins about an axis at another angular velocity, wherein the axis forms an acute angle with the incident light optical path;
an optical reader, configured to receive a reflect light returning from the target area;
a reflect polarizer set, positioned on a reflect light optical path of the reflect light, and being configured of a 90-degree polarizer; and
a processor, signally connected to the light source generator and the optical reader, and being configured to run an optical feature analysis model to extract an optical feature information from the reflect light and to determine a concentration value of a bio-substance in the target area according to the optical feature information.

2. The system according to claim 1, wherein, before determining the concentration value of the bio-substance, the optical feature analysis model further executes steps of:
reading a reflect light optical information corresponding to the reflect light, wherein the reflect light optical information comprises a reflect light intensity and a reflect light polarization information;
reading a scattering information corresponding to the target area, wherein the scattering information comprises one or multiple depolarization factors; and
performing a Mueller Matrix analysis based on the reflect light intensity and reflect light polarization information so as to obtain the optical feature information, wherein the optical feature information comprises a reflect light polarization feature and target area scattering feature.

3. The system according to claim 2, wherein the Mueller Matrix analysis is performed further based on an incident light information corresponding to the incident light, wherein the incident light information comprises a Stokes vector information, an incident light polarization information and a rotation information.

4. The system according to claim 1, wherein the target area comprises a blood vessel.

5. The system according to claim 1, wherein the bio-substance comprises blood sugar, cholesterol, lipid, protein or uric acid.

6. The system according to claim 2, wherein the one or multiple depolarization factors correspond to a 40-degree depolarization factor or a 45-degree depolarization factor.

7. The system according to claim 2, wherein the optical feature analysis model is configured to further execute steps of:
processing the Mueller Matrix by Fourier Expansion so as to generate an order coefficient formula corresponding to the reflect light intensity; and
substituting the order coefficient formula with the scattering information so as to generate an order coefficient set, thereby determining the optical feature information according to the order coefficient set.

8. The system according to claim 1, wherein the concentration value determining step further comprises: combining a stabilization time information corresponding to the optical feature information, and an average concentration information to determine the concentration value, wherein:
the stabilization time information is determined based on a pressure information applied on the target area;
the average concentration information is an average value of two or multiple initial concentration values of the bio-substance detected by the optical feature analysis model within a period defined by the stabilization time information.

9. The system according to claim 8, further comprising a pressure sensor, signally connected to the processor, and being configured to detect a pressure applied on the target area so as to produce the pressure information.

10. A system for non-invasive optical measurement of bio-substance in a biological tissue, comprising:
a light source generator, configured to generate an incident light to irradiate at a target area on an individual;
an incident polarizer set, positioned on an incident light optical path of the incident light, and being configured of a 0-degree polarizer and a rotating polarizer in a sequential manner, wherein:
the rotating polarizer spins about the incident light optical path at an angular velocity; or
the rotating polarizer spins about an axis at another angular velocity, wherein the axis forms an acute angle with the incident light optical path;
an optical reader, configured to receive a reflect light returning from the target area;
a reflect polarizer set, positioned on a reflect light optical path of the reflect light, and being configured of a 90-degree polarizer; and
a processor, signally connected to the light source generator and the optical reader, wherein:
in a training phase, the processor is configured to run a pre-optical feature analysis model to retrieve a plurality of optical feature information and a plurality of reference concentration information, and train the pre-optical feature analysis model into an optical feature analysis model based on the plurality of the optical feature information and the plurality of the reference concentration information.

11. The system according to claim 10, wherein:
before retrieving the plurality of optical feature information and the plurality of reference concentration information, the pre-optical feature analysis model executes steps of:
reading a reflect light optical information corresponding to the reflect light, wherein the reflect light optical information comprises a reflect light intensity and a reflect light polarization information;
reading a scattering information corresponding to the target area, wherein the scattering information comprises one or multiple depolarization factors;
reading a reference concentration information of a bio-substance in the target area;
performing a Mueller Matrix analysis based on the reflect light intensity and reflect light polarization information so as to obtain an optical feature information, wherein the optical feature information comprises a reflect light polarization feature and target area scattering feature;
repeat the aforementioned steps to retrieve the plurality of the optical feature information and the plurality of the reference concentration information; and
the pre-optical feature analysis model is trained by performing a regression analysis, based on a classification model, into the optical feature analysis model.

12. The system according to claim 10, wherein in an inference phase, the optical feature analysis model executes steps of:
reading a test reflect light optical information corresponding to a test reflect light, wherein the test reflect light optical information comprises a test reflect light intensity and a test reflect light polarization information;
reading a test scattering information corresponding to a test target area, wherein the test scattering information comprises one or multiple test depolarization factors;
performing a test Mueller Matrix analysis based on the test reflect light optical information and test scattering information so as to obtain a test optical feature information, wherein the optical feature information comprises a test reflect light polarization feature and a test target area scattering feature; and
determining a test concentration value of a test bio-substance in the test target area according to the test optical feature information.

13. The system according to claim 11, wherein, before obtaining the optical feature information, the pre-optical feature analysis model further executes steps of:
processing the Mueller Matrix by Fourier Expansion so as to generate an order coefficient formula corresponding to the reflect light intensity; and
substituting the order coefficient formula with the scattering information so as to generate an order coefficient set;
analyzing the order coefficient set using a correlation analytic tool including Pearson correlation coefficient, Spearman correlation coefficient analysis, Euclidean distance analysis, or cosine similarity analysis;
dividing the order coefficient set into a first coefficient set and a second coefficient set, wherein a first correlation coefficient of the first coefficient set is larger than a second correlation coefficient of the second coefficient set; and
determining the first coefficient set to be an effective feature information, thereby outputting the effective feature information to be the optical feature information.

14. The system according to claim 10, wherein the reference concentration information is retrieved by a time delay processing, comprising: detecting a plurality of delay concentration information of the bio-substance within a delay time, and processing the delay concentration information using interpolation analysis, thereby retrieving the reference concentration information.

15. The system according to claim 11, wherein the Mueller Matrix analysis is performed further based on an incident light information corresponding to the incident light, wherein the incident light information comprises a Stokes vector information, an incident light polarization information and a rotation information.

16. The system according to claim 10, wherein the target area comprises a blood vessel.

17. The system according to claim 10, wherein the bio-substance comprises blood sugar, cholesterol, lipid, protein or uric acid.

18. The system according to claim 11, wherein the one or multiple depolarization factors correspond to a 40-degree depolarization factor or a 45-degree depolarization factor.

19. The system according to claim 11, wherein the pre-optical feature analysis model performs a regression analysis based on a stabilization time information corresponding to the optical feature information, and an average concentration information, wherein:
the stabilization time information is determined based on a pressure information applied on the target area;
the average concentration information is an average value of two or multiple initial concentration values of the bio-substance detected by the optical feature analysis model within a period defined by the stabilization time information.

20. The system according to claim 19, further comprising a pressure sensor, signally connected to the processor, and being configured to detect a pressure applied on the target area so as to produce the pressure information.
